(19) United States
(12) Patent Application Publication
(10) Pub. No.: US 2015/0198586 A1
(43) Pub. Date: Jul. 16, 2015

(54) DEVICES AND METHODS FOR ENHANCED DETECTION AND IDENTIFICATION OF DISEASES

(71) Applicant: CHANG HE BIO-MEDICAL SCIENCE (YANGZHOU) CO., LTD., Yangzhou (CN)

(72) Inventors: Chang Yu, Yangzhou (CN); Xuedong Du, Yangzhou (CN)

(21) Appl. No.: 14/415,489

(22) PCT Filed: Jul. 16, 2013
(86) PCT No.: PCT/CN2013/000852

§ 371 Date: Jan. 16, 2015

Related U.S. Application Data
(60) Provisional application No. 61/672,231, filed on Jul. 16, 2012.

(51) Int. Cl. G01N 33/50

(52) U.S. Cl. CPC G01N 33/5091

(57) ABSTRACT

Disclosed are micro-devices and methods of using the same for detecting at the microscopic level a property of a biological material contained in a liquid or existing in a liquid state. The device comprises an inlet (0212, 0322) for the biological material to enter the micro-device, an optional pre-treatment unit, a probing unit (0325), a detection unit (0326), a system controller, and an exit (0213, 0323) for the residual biological material or waste to be ousted from the micro-device. The micro-device can be provided at least one chemical, biological, or bio-chemical additive (0422) in conjunction with the micro-device to enhance detection sensitivity and specificity.

### CROSS-REFERENCE OF RELATED APPLICATION

This application claims priority to U.S. Application No. 61/672,231, filed on Jul. 16, 2012, the contents of which are incorporated herein by reference in their entireties.

### BACKGROUND OF THE INVENTION

Many serious diseases with high morbidity and mortality, including cancer and heart diseases, are very difficult to diagnose early and accurately. Current disease diagnosis technologies typically rely on macroscopic data and information such as body temperature, blood pressure, and scanned images of the body. To detect serious diseases such as cancer, many of the diagnosis apparatus commonly used today are based on imaging technologies, including x-ray, CT scan, and nuclear magnetic resonance (NMR). While they provide various degrees of usefulness in disease diagnosis, most of them cannot provide accurate, totally safe, and cost-effective diagnosis of such serious diseases as cancer at an early stage. Further, many of the existing diagnosis techniques and related apparatus are invasive and sometimes not readily accessible, especially in remote regions or rural areas.

Even the newly emerged technologies such as those deployed in DNA tests have not been proven effective in diagnosing a wide range of diseases in a rapid, reliable, accurate, and cost-effective manner. In recent years, there have been some efforts in using nano technologies for various biological applications, with most of the work focused on gene mapping and moderate developments in the field of disease detection. For instance, Pantel et al. discussed the use of a MicroEelectroMechanical Systems (MEMS) sensor for detecting cancer cells in blood and bone marrow in vitro (see, e.g., Klaus Pantel et al., Nature Reviews, 2008, 8, 329); Kubena et al. disclose in U.S. Pat. No. 6,922,118 the deployment of MEMS for detecting biological agents; and Weissman et al. disclose in U.S. Pat. No. 6,330,885 utilizing MEMS sensor for detecting accretion of biological matter.

However, to date, most of the above described technologies have been limited to isolated examples for sensing, using systems of relatively simple constructions and large dimensions but often with limited functions, and lack sensitivities and specificities. Further, some existing technologies utilizing nano-particles and biological approaches have the drawbacks of requiring complicated sample preparation procedures (such as using chemical or biological markers), difficulty in data interpretation, and too much reliance on visual and color change as means of diagnosis (which is subjective and of limited resolution), making them unsuitable for early stage disease detection, e.g., for such serious diseases as cancer, and particularly for routine hospital screening and/or regular physical check-up examinations. Some cannot achieve high degree of sensitivity and specificity simultaneously.

These drawbacks call for novel solutions that not only overcome them but also bring improved accuracy, sensitivity, specificity, efficiency, non-invasiveness, practicality, simplicity, and speed in early-stage disease detection at reduced costs.

The existing detection technology and equipment are dominated by single-technology based single purpose equipment with limited disease detection coverage scope, limited functionalities and low efficiency. They are often very extensive, with large foot print (such as NMR, CT, and x-ray machine). They mainly consist of three large groups: (a) imaging-based technology for mid to late stage cancers, (b) bio-marker based technology which offers some sensitivity to specific type of cancer (but for a given bio-marker, it is typically only sensitive to one type or one sub-type of cancer, with relatively low level of specificity), and (c) genomics based detection technology which is relatively insensitive and long processing time.

Because the images are able to identify the disease only when it is in the mid to late stage, the methods and apparatus that heavily depend on imaging-based technologies are not suitable or capable of detecting early-stage diseases, particularly cancer.

Compared with imaging based technologies, bio-marker can detect certain specific cancer at an earlier stage. However, it is a complicated detection technology and process. With a relatively low specificity, it is prone to false alarm in detection. Further, it is narrow in detection scope and applications in terms of cancer types, since typically for a given bio-marker, it is only sensitive to a particular type or sub-type of cancer. As a result, it may not be suited for a general physical check-up (such as annual physical) for cancer screening. It also may not be used alone for cancer detection and it may require additional diagnosis tools for verifications.

Some other techniques may be capable of detecting certain general parameters of cancer, but they cannot distinguish or identify (i.e., determine) the specific type of cancer. In other words, even if those techniques can alert the existing of a cancerous disease, it cannot specify the type of cancer and hence requires additional diagnosis using other detection technologies. Thus, it alone cannot offer a cancer diagnosis solution.

There is a need for providing the ability in terms of both general (cancer detection at an early stage) and specific type(s) of cancer. The limitations described above on the currently existing cancer detection technologies show that no currently existing methods and equipments can effectively detect simultaneously both general parameters in a biological entity for detecting of cancer and identifying the specific cancer type.

### SUMMARY OF THE INVENTION

The present invention in general relates to a class of innovative and integrated micro-devices for carrying out much enhanced disease detection and identification at microscopic levels, in vivo or in vitro, on a single cell, a single biological molecular (e.g., DNA, RNA, or protein), a single biological subject (e.g., a single virus), or other sufficiently small unit or fundamental biological composition. This class of micro-devices can be made by using state-of-the-art micro-device fabrication technologies and novel process flows such as integrated circuit fabrication technologies. As used herein, the term “disease detection micro-device” can be interchanged with such terms as disease detection device or apparatus integrated with micro-devices, or any other similar terms of the same meaning. The micro-devices of this invention contain multiple micro units to perform different functions and optionally detect multiple parameters of a biological subject to be detected or analyzed. Optional components of the apparatus includes means to perform at least the function of addressing, controlling, forcing, receiving, amplifying, manipulating, processing, analyzing, making decisions (e.g., logic decisions), or storing information from each probe. Such means can be, e.g., a central control unit that includes a controlling circuitry, an addressing unit, an amplifier circuitry, a logic processing circuitry, an analog device, a memory unit, an application specific chip, a signal transmitter, a signal receiver, or a sensor.

These disease detection micro-devices are capable of detecting diseases at their early stages with a higher and much improved degree of sensitivity, specificity, speed, simplicity, practicality, convenience (e.g., simpler operating procedures or reduced apparatus size), or affordability (e.g., reduced costs), with substantially reduced to no invasiveness and side effects. Accordingly, the micro-devices of this invention are capable of perform at a much higher level than those of conventional disease detection apparatus or technologies.

Examples of inventive fabrication techniques or processes that can be used to make the micro-devices of this invention include, but are not limited to, mechanical, chemical, physical-chemical, chemical mechanical, electrical, physical, magnetic, bio-chemical, bio-physical, electro-magnetic, bio-physical mechanical, electro-mechanical, bio-electro-mechanical, micro-electro-mechanical, electro-chemical-mechanical, electro-bio-chemical-mechanical, nano-fabrication techniques, integrated circuit and semiconductor manufacturing techniques and processes. For a general description of some of the applicable fabrication technologies, see, e.g., R. Zaouk et al., Introduction to Microfabrication Techniques, in Microfluidic Techniques (S. Minteer, ed.), 2006, Humana Press; Microsystem Engineering of Lab-on-a-chip Devices, 1st Ed. (Geschke, Klank & Telleman, eds.), John Wiley & Sons, 2004. Micro-device functionalities would at least include sensing, detecting, measuring, diagnosing, monitoring, and analyzing for disease diagnosis. Multiple micro-devices can be integrated onto a piece of detection apparatus to make the apparatus more advanced and sophisticated for further enhanced measurement sensitivity, specificity, speed and functionalities, with ability to measure the same parameter or a set of different parameters.

In one aspect, the invention provides micro-devices for detecting at the microscopic level a property of a biological material contained in a liquid or existing in a liquid state, comprising, an inlet for the biological material to enter the micro-device, an optional pre-treatment unit, a probing unit, a detection unit, a system controller, and an exit for the residual biological material or waste to be ousted from the micro-device.

In some embodiments, the property to be detected comprises a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical signal, or a combination thereof.

The electrical property can be surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property can be temperature or vibrational frequency; the optical property can be optical absorption, optical transmission, optical reflection, optical-electrical property, brightness, or fluorescent emission; the chemical property can be pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property can be density, shape, volume, or surface area; the biological property can be surface shape, surface area, surface charge, surface biological property, surface chemical property, pH, electrolyte, ionic strength, resistivity, cell concentration, property relating to a bio-marker, or biological, electrical, physical or chemical property of solution; the acoustic property can be frequency, speed of acoustic waves, acoustic frequency and intensity spectrum distribution, acoustic intensity, acoustical absorption, or acoustical resonance; the mechanical property can be internal pressure, hardness, flow rate, viscosity, shear strength, elongation strength, fracture stress, adhesion, mechanical resonance frequency, elasticity, plasticity, or compressibility. The above stated properties can be static or dynamic and changing.

In some embodiments, each micro-device comprises one channel or multiple channels, micro-pumps, inlet opening and outlet opening, wherein signal detection unit(s) and optionally probing unit(s) are located at the walls.

In some other embodiments, each micro-device can further include a capillary tube having two terminal openings and a sidewall with an interior surface, an outer surface, and optionally a micro-pump, a sample pre-treatment unit, a sample re-circulation unit, and a sample discharge unit, wherein one of the two terminal openings is the inlet of the micro-device and the other terminal opening is the outlet of the micro-device, and the capillary tube houses the detection unit and optionally probing unit.

In some examples, the capillary tube includes an interior core and an interior channel which is defined by the interior core and the interior surface of the capillary tube's sidewall.

In some other examples, the capillary tube comprises one or more pin-holes each of which runs through the exterior and interior surfaces of the capillary tube's sidewall and houses a probing unit or a detecting unit.

In some other examples, the capillary tube comprises at least two pin-holes each of which penetrates through the exterior and interior surfaces of the capillary tube's sidewall and houses a probing unit or a detecting unit.

In some other embodiments, each of the probing unit or detecting unit is capable of sending a probing signal or measure at the microscopic level a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical signal, or a combination thereof. For example, the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility. The above stated properties can be static or dynamic and changing.

Each pin-hole can be fabricated by a method comprising mechanical, electric, magnetic, electro-magnetic, radio-active, ionic, thermal, optical, acoustical, chemical, electro-mechanical, electro-chemical, and electro-chemical-mechanical treatments or technologies.

Each pin-hole can have a diameter or width ranging from 0.01 micron to 1 centimeter, with a preferred range from 10 microns to 2,000 microns.

The capillary tube can have a circular, elliptical, square, rectangular, triangular, or polygonal shape.

In some embodiments, the capillary tube has an inner diameter or width ranging from about 0.1 um to about 10 mm, from about 20 microns to about 300 microns, from about 0.1 micro to about 100 microns, or from about 5 um to about 500 urn. On the other hand, the capillary tube can have an a length ranging from 100 um to about 100 mm or from 100 um to about 100 mm.

In some embodiments, the probing unit and the detection unit are embodied in one unit.

In some embodiments, the probing unit applies a radioactive probing signal to the biological material.

In some embodiments, the probing unit comprises a radioactive element that generates the radioactive probing signal. The radioactive element can include, e.g., H-3, Be-7, Na-22, Ca-46, Fe-55, Fe-59, Co-56, Co-57, Co-58, Co-60, Ni-63, Zn-65, Zn-72, Kr-85, Sr-89, Sr-90, Y-90, Y-91, Zr-94, Nb-93, Nb-95, Mo-93, Ru-103, Ru-106, Ag-111, Sb-125, Te-127, Te-129, I-123, I-131, Xe-125, Xe-127, Xe-133, Cs-134, Cs-137, Ce-144, Pm-147, Eu-154, Eu-155, Ir-188, Ir-189, Ir-190, Ir-192, Ir-192, Ir-193, Ir-194, Ir-194, Pb-210, Po-210, Rn-220, Rn-222, Ra-224, Ra-225, Th-228, Th-229, Th-232, Th-234, Pa-234, Pu-238, Pu-239, Pu-240, Pu-241, Am-241, Am-242, Cm-242, Cm-243, and Cm-244. Na-22 and Co-60 are commonly used.

In some embodiments, the probing unit comprises a positron emitting device that delivers the probing signal to the biological material.

In some embodiments, the detecting unit comprises a spectroscopic collector integrated to the detecting unit. Examples of the spectroscopic collectors include photo detector which has a high sensitivity of detecting photons, acoustic transducers, detectors such as fringing effect based transducers for electrical and/or vibrational signal detections, analyzers for chemical, biological, and bio-chemical component analysis such as high-performance liquid chromatography (HPLC), ion-coupled mass spectroscopy, and mass spectroscopy.

In some embodiments, the micro-device further includes an additive inlet for introducing an additive to the liquid containing the biological material.

In some embodiments, at least an additive inlet is connected to a pin-hole. In some other embodiments, at least one additive inlet is located at the probing unit or the detection unit.

In some embodiments, the additive communicates with, interacts with, or probes the biological material; or the additive triggers, participates in, or functions in a response by the biological material (e.g., at the cellular level); or enhances the measurement signal of the tested property of the biological material. The biological material's response to the proving signal can be a reaction or chain reaction within itself, therefore increasing the strength of a microscopic property or thereby increasing the signal/noise ratio.

In some embodiments, the additive reacts or binds with the biological material to form a complex, conjugate, or aggregate, thereby increasing the strength of the property of the biological material that is to be tested.

In some embodiments, the additive selectively reacts or binds with the biological material or a component thereof, to form a complex, conjugate, or aggregate, thereby selectively increasing the strength of the property of the biological material or a component thereof that is to be tested.

In some embodiments, the additive comprises a liquid solution, solid nanoparticles, or gas.

In some embodiments, the liquid solution is an aqueous solution or an organic solution and comprises potassium permanganate, glucose, glucose compounds, hydrogen phosphate, pyruvate acid, sodium pyruvate, bromide pyruvate, bromopyruvic acid, acetic acid, propionaldehyde, glycerldehyde, methylglyoxal, lactate dehydrogenase, alanine, lactic acid, amino acid, a protein, calcium, potassium, sulfur, sodium, magnesium, copper, zinc, selenium, molybdenum, fluorine, chlorine, iodine, manganese, cobalt, iron, or an enzyme.

In some embodiments, the enzyme comprises a hexokinase (e.g., pyruvate carboxylase or PEP carboxylinase).

In some embodiments, the gas components or liquid solutions comprise O₂, O₃, CO, CO₂, calcium, sodium, potassium, sulfur, sodium, magnesium, copper, zinc, selenium, molybdenum, fluorine, chlorine, iodine, manganese, cobalt, iron, or carbon based organic groups including but not limited to organometallic compound group, aldehyde (carbonyl group), ketone (carbonyl group), carboxylic acid (carboxyl group), amine (amino group), amino acid (amino group plus carboxyl group) and alcohol (hydroxyl group).

In some embodiments, the additive comprises an ion, oxidant, a reductant (a reducing agent), or a bio-active compound. As used herein, the term “bio-active compound” refers to a compound used or involved in cellular functions and processes such as metabolic processes. Examples of the bio-active compound include carbohydrates, proteins and enzymes.

Examples of suitable ions include, but are not limited to, Fe³⁺, Fe²⁺, Ag⁺, Cr³⁺, Na⁺, K⁺, Pt²⁺, Mg²⁺, H⁺, Ca²⁺, Hg²⁺, Al³⁺, NH₄⁺, H₃O⁺, Hg₂⁴⁺, Cl⁻, F⁻, Br⁻, O²⁻, CO₃²⁻, HCO₃⁻, OH⁻, NO₃⁻, PO₄³⁻, SO₄²⁻, CH₃COO⁻, HCOO⁻, C₂O₄²⁻, and CN⁻.

Examples of suitable oxidant include, but are not limited to, oxygen, ozone, hydrogen peroxide, an inorganic peroxide, nitric acid, a nitrate compound, a chromium compound, a permanganate compound, sulfuric acid, persulfuric acid, fluorine, chlorine, bromine, iodine, chlorite, chlorate, perchlorate, other analogous halogen compounds (for example, 4-chlorotoluene, dibromopentane, bromoethane, 2-chloropropane, fluorocyclopentane, and 2-iodo-2-methylpentane), hyperchlorite, other hypohalite compounds (for example, hypoiodous acid, hypobromite, hypochlorite, and hypofluorous acid), sodium perborate, nitrous oxide, sliver oxide, osmium tetroxide, Tollens' reagent, 2,2′-dipyridyldisulfide, urea, and their combinations and their compounds (for example, silver nitrate, ferric nitrate, urea nitrogen, blood urea nitrogen, and potassium permanganate).

Examples of suitable reductant include, but are not limited to, nascent hydrogen, a compound containing Fe cation (e.g., FeSO₄), sodium amalgam, sodium borohydride, a sulfite compound, hydrazine, a compound containing the Sn²⁺ ion, zinc-mercury amalgam, lithium aluminum hydride, Lindlar catalyst, formic acid, oxalic acid, ascorbic acid, phosphites, hypophosphites, and phosphorous acid. Examples of suitable bio-active compound include, but are not limited to, glucose, fructose, pyruvate, galactose, amino acid, acetic acid, glyoxylic acid, oxalic acid, propionic acid, acetic acid, enzyme (oxidoreductases (dehydrogenase, luciferase, DMSO reductase), transferases, hydrolases, lyases, isomerases, ligases, RNA-enzyme, DNA polymerase, RNA polymerase, aminoacyl tRNA synthetases, and ribosomes), artificial enzyme (for example, scaffolded histidine residues), and enzymes with their respective cofactors.

In some embodiments, the detection unit detects the property at the microscopic level and generates a machine-readable tested signal.

In some embodiments, the system controller processes the machine-readable signal to generate data that can be displayed or readable by human eyes.

In some embodiments, the system controller includes a first A/D converter, a computer, and a data display device. For instance, the display device can be a computer screen or a printer.

In some embodiments, the system controller further includes an amplifier which amplifies the machine-readable tested signal before it reaches the A/D converter and then the computer. The amplifier is optionally a lock-in amplifier.

In some embodiments, the computer includes a CPU, a RAM, and a ROM. The CPU (i.e., central processing unit (CPU) is the hardware that carries out the instructions of a computer program by performing the basic arithmetical, logical, and input/output operations of the system. The RAM (i.e., random access memory) is a form of computer data storage. The ROM (i.e., read-only memory) is a storage medium.

In some embodiments, the system controller further includes a manipulator which initiates or generates a disturbing signal and then sends it to the computer for processing before the disturbing signal is applied to the biological material by the disturbing unit. The disturbing signal can be a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical signal, or a combination thereof.

For example, the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility. The above stated properties can be static or dynamic and changing.

In some embodiments, the system controller further includes a second A/D (i.e., alternative/direct) converter and a signal generator which process the disturbing signal after the computer but before the disturbing signal is applied to the biological material by the disturbing unit.

In some embodiments, the pretreatment unit separates the biological material into different components by the difference in a common property of the biological material. Optionally, the pretreatment unit can treat biological material such as surface treatment. Another optional pretreatment includes sequential addition of desired biological, bio-chemical or chemical components to the biological material at desired time interval and/or temperature to be tested. For example, an oxidizer such as hydrogen peroxide can be added to a biological sample to be tested first, followed by addition of a second oxidizer. In another example, an oxidizer can be added to a biological sample to be tested first, followed by the addition of a catalyst. In yet another example, an oxidizer can be added to a biological sample to be tested first, followed by the addition of a catalyst, and finally, an enzyme is added to the mixture.

In some embodiments, a micro-device of this invention further comprises a second optional pre-treatment unit, a second probing unit, and a second detection unit, which form a second stage of detection within the micro-device, wherein the second stage of detection detects the same or different property at the microscopic level as the previous stage. The second stage can differ from the first stage, e.g., by its geometry (e.g., width, height, length, or shape of the channel) or the probing unit or detection unit (thereby the property to be detected). The geometry information, probing signals applied by the probing units, the signals detected and measured by the detection units, and the optionally use of one or more enhancers, results in enhanced specificity and sensitivity of the detection and differentiation of different types of disease.

Another aspect of this invention provides methods for enhancing the detection or identification of a disease in a biological subject to be screened. Each of the methods includes the steps of:

taking a biological material sample from the biological subject to be screened,

preparing a liquid solution of the biological material sample (including converting the biological material sample into a liquid state),

injecting the biological sample's liquid solution to a micro-device,

adding a bio-identifier to the liquid solution before the injection or when the liquid solution is inside the micro-device;

optionally, adding at least one additional biological, chemical, or bio-chemical component into the liquid solution for measurement sensitivity enhancement;

detecting and measuring, at the microscopic level, a property of the biological sample in the liquid solution; and

comparing the measured property with that of a biological subject free of the disease.

As used herein, the terms “bio-identifier,” “enhancer,” and “additive” are interchangeable. They comprise an ion, an oxidizer or oxidant, a reducing agent, an inhibitor, a catalyst, an enzyme, a bio-marker, a chemical-marker, a bio-chemical marker, a chemical component, a bio-chemical component, a biological component, an organic component, a metal-organic component, a bio-chemical component, an optical component, a florescence component, a protein, a virus, a coloring agent, an antibody, or a combination thereof. As used herein, the term “or” is meant to include both “and” and “or.” In other words, the term “or” may also be replaced with “and/or.”

In some embodiments, the biological material sample is a DNA, telomere of DNA, RNA, chromosome, cell, cell substructure, protein, tissue, virus, blood, urine, sweat, tear, saliva, or organ tissue.

In some embodiments, the liquid solution is an aqueous solution or a solution in an organic solvent.

In some embodiments, the disease is a cancer.

In some embodiments, the cancer is bladder cancer, breast cancer, colon cancer, rectal cancer, endometrial cancer, kidney cancer, leukemia cancer, lung cancer (including bronchus), melanoma cancer, non-Hodgkin lymphoma, pancreatic cancer, prostate cancer, or thyroid cancer.

In some embodiments, the property to be detected and measured is a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical property, or a combination thereof.

For example, the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility. The above stated properties can be static or dynamic and changing.

In some embodiments, the bio-identifier exists as a liquid solution, solid nanoparticles, or gas.

In some embodiments, the liquid solution is an aqueous solution or an organic solution and comprises potassium permanganate, glucose or a glucose compound, hydrogen phosphate, pyruvate acid, sodium pyruvate, bromide pyruvate, bromopyruvic acid, acetic acid, propionaldehyde, glycerldehyde, methylglyoxal, lactate dehydrogenase, alanine, lactic acid, amino acid, a protein, calcium, potassium, sulfur, sodium, magnesium, copper, zinc, selenium, molybdenum, fluorine, chlorine, iodine, manganese, cobalt, iron, or an enzyme.

In some embodiments, the enzyme comprises a hexokinase (e.g., pyruvate carboxylase and PEP carboxylinase), oxidoreductases (dehydrogenase, luciferase, DMSO reductase), transferases, hydrolases, lyases, isomerases, ligases, RNA-enzyme, DNA polymerase, RNA polymerase, aminoacyl tRNA synthetases, and ribosomes, artificial enzyme (for example, scaffolded histidine residues), and enzymes with their respective cofactors.

In some embodiments, the gas and liquid solution comprises O₂, O₃, CO, CO₂, calcium, sodium, potassium, sulfur, sodium, magnesium, copper, zinc, selenium, molybdenum, fluorine, chlorine, iodine, manganese, cobalt, iron, or carbon based organic groups including but not limited to organometallic compound group, aldehyde (carbonyl group), ketone (carbonyl group), carboxylic acid (carboxyl group), amine (amino group), amino acid (amino group plus carboxyl group) and alcohol (hydroxyl group).

In some embodiments, wherein the additive comprises an ion, an oxidant, a reductant, or a bio-active compound. Suitable examples of ion include Fe³⁺, Fe²⁺, Ag⁺, Cr³⁺, Na⁺, K⁺, Pt²⁺, Mg²⁺, H⁺, Ca²⁺, Hg²⁺, Al³⁺, NH₄⁺, H₃O⁺, Hg₂⁴⁺, Cl⁻, F⁻, Br⁻, O²⁻, CO₃²⁻, HCO₃⁻, OH⁻, NO₃⁻, PO₄³⁻, SO₄²⁻, CH₃COO⁻, HCOO⁻, C₂O₄²⁻, and CN⁻. Suitable examples of oxidant include, but are not limited to, oxygen, ozone, hydrogen peroxide, an inorganic peroxide, nitric acid, a nitrate compound, a chromium compound, a permanganate compound, sulfuric acid, persulfuric acid, fluorine, chlorine, bromine, iodine, chlorite, chlorate, perchlorate, other analogous halogen compounds (for example, 4-chlorotoluene, dibromopentane, bromoethane, 2-chloropropane, fluorocyclopentane, and 2-iodo-2-methylpentane), hyperchlorite, other hypohalite compounds (for example, hypoiodous acid, hypobromite, hypochlorite, and hypofluorous acid), sodium perborate, nitrous oxide, sliver oxide, osmium tetroxide, Tollens' reagent, 2,2′-dipyridyldisulfide, urea, and their combinations and their compounds (for example, silver nitrate, ferric nitrate, urea nitrogen, blood urea nitrogen, and potassium permanganate). Suitable examples of reductant include nascent hydrogen, a compound containing Fe²⁺ ion (e.g., FeSO₄), sodium amalgam, sodium borohydride, a sulfite compound, hydrazine, a compound containing the Sn²⁺ ion, zinc-mercury amalgam, lithium aluminum hydride, Lindlar catalyst, formic acid, oxalic acid, ascorbic acid, phosphites, hypophosphites, or phosphorous acid. Suitable examples of the bio-active compound include glucose, fructose, pyruvate, galactose, amino acid, acetic acid, glyoxylic acid, oxalic acid, propionic acid, acetic acid, and a certain enzyme. Suited examples of enzyme include but not limited to oxidoreductases (dehydrogenase, luciferase, DMSO reductase), transferases, hydrolases, lyases, isomerases, ligases, RNA-enzyme, DNA polymerase, RNA polymerase, aminoacyl tRNA synthetases, and ribosomes, artificial enzyme (for example, scaffolded histidine residues), and enzymes with their respective cofactors.

In some embodiments, a method of this invention further includes mixing the biological subject to be tested with an additive before the detection to enhance the sensitivity and/or specificity of the detection.

In some embodiments, a method of this invention further includes mixing the biological subject to be tested with an additive during the detection to enhance the sensitivity and/or specificity of the detection, during which the dynamic information in interaction between the biological subject to be tested and the additive is obtained.

Still in some other embodiments, a method of this invention further includes mixing the biological subject to be tested with at least two additives, either together or separately, before or during the detection or both.

In some embodiments, the bio-identifier or additive comprises a chemical additive, a bio-chemical additive, a biological additive, a solid particle, or a nano-particle with a high surface area.

In some embodiments, mixing of the biological material to be tested with the additive results in one or multiple reactions between the biological subject to be tested and the additive, or among the biological subject to be tested, other component(s) in the liquid solution, and the additive. The reaction may include an oxidation, reduction, catalytic, chemical, biological, bio-chemical, bio-physical, bio-mechanical, bio-optical, bio-electrical, electro-optical, bio-thermal, bio-electro-optical, bio-electro-mechanical, exothermic, or chain reaction.

In some instances, the reaction is or causes a chain reaction in which a signal (particularly a weak signal) to be detected can be amplified, thereby enhancing the detection sensitivity and specificity, e.g., for disease such as one or more types of cancer, or for differentiating different types of disease.

In some other instances, the reaction will enhance the sensitivity of detecting oxygen level in the biological subject to be tested.

In some embodiments, the bio-identifier or additive comprises an oxidant, a reductant, an inhibitor, a catalyst, an enzyme, a protein, a virus, a coloring agent, a bio-marker, a chemical-marker, an organic compound, a metal-organic compound, an antibody, a bio-chemical-marker, chemical, bio-chemical, a biological component, thermal material, and an optical material including fluoresce materials.

In the methods of this invention, the additives or bio-identifiers can be added to the biological sample to be detected at the same time or different times (with different or same time interval).

As a first example, they can be added in the following sequence: adding an oxidizer to the liquid solution containing the biological subject to be tested first; optionally adding an catalyst; optionally adding a bio-chemical additive; optionally adding an inhibitor; optionally adding a bio-marker; optionally adding a chemical; optionally adding an enzyme; and optionally adding a reducing agent.

As a second example, the additives or bio-identifiers are added in the following sequence: adding a catalyst to the liquid solution containing the biological subject to be tested first; optionally adding an oxidizer; optionally adding a bio-chemical additive; optionally adding an inhibitor; optionally adding a bio-marker; optionally adding a chemical; optionally adding an enzyme; and optionally adding a reducing agent.

As a third example, the additives or bio-identifiers are added in the following sequence: adding a bio-chemical additive to the liquid solution containing the biological subject to be tested first; optionally adding an catalyst; optionally adding a reducing agent; optionally adding an inhibitor; optionally adding a bio-marker; optionally adding a chemical; optionally adding an enzyme; and optionally adding an oxidizer.

As a fourth example, the additives or bio-identifiers are added in the following sequence: adding a reducing agent to the liquid solution containing the biological subject to be tested first; optionally adding an catalyst; optionally adding a bio-chemical additive; optionally adding an inhibitor; optionally adding a bio-marker; optionally adding a chemical; optionally adding an enzyme; and optionally adding an oxidizer.

As a fifth example, the additives or bio-identifiers are added in the following sequence: adding to a nano-particle dispersion an additive selected from a group comprising of an oxidizer, a reducing agent, an inhibitor, a catalyst, an enzyme, a protein, a virus, a coloring agent, a bio-marker, a chemical-marker, an organic compound, a metal-organic compound, an antibody, a bio-chemical-marker, chemical, bio-chemical, a biological component, a thermal material, and an optical material including fluoresce materials; mixing the above dispersion well; optionally processing the above dispersion at a desired temperature for a desired time; and adding the above dispersion to a liquid phase solution containing a biological subject to be tested.

As a sixth example, the additives or bio-identifiers are added in the following sequence: adding to a nano-particle dispersion an additive selected from a group comprising of an oxidant, a reductant, an inhibitor, a catalyst, an enzyme, a protein, a virus, a coloring agent, a bio-marker, a chemical-marker, an organic compound, a metal-organic compound, an antibody, a bio-chemical-marker, a chemical, a bio-chemical, a biological component, a thermal material, and an optical material including fluoresce materials; mixing the above dispersion well; optionally processing the above dispersion at a desired temperature for a desired time; and adding a liquid phase solution containing a biological subject to be tested to the above dispersion.

In some embodiments of the methods of this invention, the additive or bio-identifier can include an oxidizer, a reducing agent, an inhibitor, a catalyst, an enzyme, a protein, a virus, a coloring agent, a bio-marker, a chemical-marker, an organic compound, a metal-organic compound, an antibody, a bio-chemical-marker, chemical, bio-chemical, a biological component, a thermal material, and an optical material including fluoresce materials. Examples of the catalyst include an enzyme, an ion, a biological component, a chemical component which speeds up reactions, or a combination thereof.

In some other embodiments, the additives are pre-added to the biological samples to be tested before being introduced into the micro-device for detection.

In some other embodiments, the additives are added to the micro-device through separate inlet and mixed with the biological samples to be tested in the micro-device before detection.

In yet some other embodiments, the additives are added to the micro-device through separate inlets and mixed with the biological samples to be tested in the micro-device during detection.

In some embodiments, a property of the biological subject is measured at the microscopic level using the micro-device after the biological subject is mixed with the additive or additives. The property to be measured can include a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical property, or a combination thereof.

For example, the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility.

The methods of this invention not only allows detection of the existence of disease in the biological subject by differentiating normal biological material from diseased biological material, but also obtaining information on the type or types of the disease thereby differentiating the different types of disease (e.g., cancer). The differentiation of different types of disease can be based in part on the geometry of the detection unit, probing signal, change in the probing signal, and/or the biological sample; or on the cell surface properties, cell membrane properties, oxygen level, oxygen location, oxygen bonding, electric charge density, electric charge location, or dynamic properties of the biological sample. Examples of the cell surface or cell membrane properties include surface absorption and adsorption ability of the biological sample, the oxygen level, oxygen location, oxygen bonding on the cell surface or membrane, ion concentration, ion gradient, membrane resting potential, cell surface charge, or the permeability and transportation ability of the membrane. The above stated properties can be static or dynamic and changing.

In some embodiments, the additives or bio-identifiers include an oxidizer, an enzyme, a reducing agent, an inhibitor, a bio-marker, a bio-chemical component, a chemical component, a biological component, a protein, a virus, a thermal component, an optical component, a fluoresce material, or a catalyst, which are added to the biological at different times before the detection.

In some embodiments, the additives or bio-identifiers include at least a bio-active compound (e.g., a protein that binds the biological material).

In some embodiments, at least two additives or bio-identifiers are mixed before they are added to the biological sample to be detected.

In some embodiments, the complex of the biological sample and additives is separated before being detected by the detection unit.

A detection method of this invention can further include the following steps:

scanning the range of a probing signal,

collecting one or more response signals from the biological sample being tested,

analyzing the one or more response signals from the biological sample being tested as a function of the scanned value of the probing signal, and

making conclusion or recommendation on whether there is a disease and the type of disease.

In some embodiments, the probing signal or each of the one or more response signal is a signal of thermal, optical, acoustical, biological, chemical, radioactive, electrical, magnetic, electro-mechanical, electro-chemical, electro-magnetic, electro-optical, electro-thermal, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical signal, or a combination thereof. For example, the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility. The above stated properties can be static or dynamic and changing.

In some embodiments, the analysis of the response signals includes plotting curves specifically characteristic of a disease (e.g., cancer).

In some embodiments, the probing signal applied to the biological sample to be tested is based on an acoustical signal, a laser beam, and is scanned across its frequency range and intensity range, and the response signals from the biological sample being tested are then measured.

In some other embodiments, the probing signal applied to the biological sample to be tested is based on an applied mechanical force and scanned across its magnitude of the force, and the response signals from the biological sample being tested is then measured.

In still some other embodiments, the probing signal applied to the biological sample to be tested is based on a thermal energy and scanned across its temperature range and energy level, and response signals from the biological sample being tested are then measured.

In yet still some other embodiments, the probing signal applied to the biological sample to be tested is based on an electrical voltage (e.g., pulsed electrical voltage) and scanned across its voltage range, and the response signals from the biological sample being tested are then measured.

The micro-devices and methods of this invention can result in a higher degree of sensitivity for and specificity of the disease to be detected than a method without the application of the bio-identifier.

### BRIEF DESCRIPTIONS OF THE FIGURES

FIG. 1 shows the diagram of an apparatus of this invention for detecting disease, and a system controller contained in the apparatus.

FIG. 2 illustrates an example of capillary tube which can be included in an apparatus of this invention.

FIG. 3 illustrates another example of capillary tube, with optional probing and detection units, which can be included in an apparatus of this invention.

FIG. 4 shows the diagram of another apparatus of this invention for detecting disease and how additives enhance the measurement of microscopic property of the biological material.

FIG. 5 illustrates how bio-identifiers enhance the measurement of microscopic property of the biological material.

FIG. 6 illustrates how the method of this invention which uses bio-identifiers to enhance the measurement of microscopic property of the biological material, improves the sensitivity and specificity of the detection of disease.

FIG. 7 further illustrates how bio-identifiers enhance the measurement of microscopic property of the biological material.

FIG. 8 illustrates how the method of this invention which uses bio-identifiers improves the sensitivity and specificity of the detection of disease.

FIG. 9 shows the detection of a control group and of an ovarian cancer group by the invention disclosed herein.

FIG. 10 shows the detection of a control group and of a liver cancer group by the invention disclosed herein.

FIG. 11 shows the different in detecting and identifying normal group and liver cancer group, with and without an additive, of the invention disclosed herein.

FIG. 12 shows the different detection specificity and accuracy of the invention disclosure herein for detecting and differentiating liver cancer and ovarian cancer, with and without an additive.

FIG. 13 shows the effectiveness of the invention disclosed herein in monitoring the post-chemotherapy recurrence of breast cancer.

FIG. 14 shows the effectiveness of the invention disclosed herein in monitoring the post-radiotherapy recurrence of gastric cancer.

FIG. 15 shows the effectiveness of the invention disclosed herein in monitoring the post-chemotherapy recurrence of gastric cancer.

### DETAILED DESCRIPTION OF THE INVENTION

In one aspect, the present invention provides micro-devices for detecting at the microscopic level a property of a biological material contained in a liquid or existing in a liquid state, comprising, an inlet for the biological material to enter the micro-device, an optional pre-treatment unit, a probing unit, a detection unit, a system controller, and an exit for the residual biological material or waste to be ousted from the micro-device.

The probing unit and the detection unit each can be fabricated by methods previously developed by the same inventors and described in earlier applications. See, e.g., WO 2011/103041 and WO 2011/005720, the contents of which are incorporated herein by reference in their entireties.

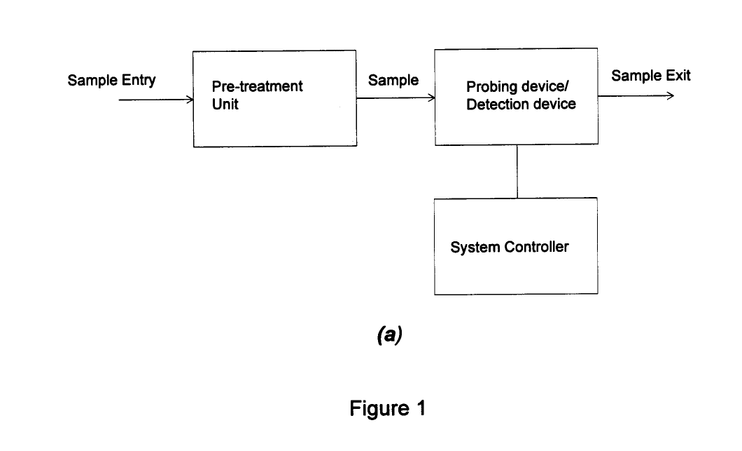

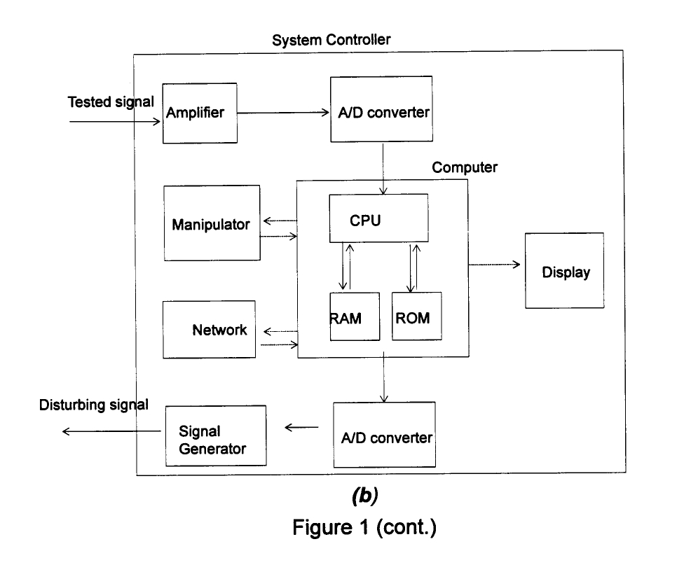

FIG. 1 shows an example of the micro-devices of this invention for detecting at the microscopic level a property of a liquid or dissolved solution (e.g., food, beverage, oil, chemical, drug, blood, urine, sweat, saliva, and other biological liquid). FIG. 1(a) illustrates a micro-device with at least a sample entry, a sample exit, a pre-treat unit, a probing device, a detection device, and a system controller. FIG. 1(b) illustrates a system controller's diagram. In the system, the tested signal is collected by the system controller through amplifier and converter. It is then processed and analyzed by the computer. The analyzed result is transmitted to a recorder or displayed on a display device. The disturbing (probing) signal is initiated by operator through manipulator. It is then processed by the computer, convertor, and then produced by the signal generator, then being applied to the objects to be measured.

In one embodiment, the micro-devices comprise a biological sample pre-treatment unit in which diseased biological items (such as circulating tumor cells) are concentrated, an inlet for bringing in bio-identifier, single channels in which biological sample can flow through, multiple channels in which biological sample can flow through, a detection probe unit for sending disturbing signals, a detection detector unit for sensing response signals, or an outlet for biological sample to flow out. The sample pre-treatment unit has one stage or multiple stages (which comprise filtration, electrophoresis, bio-marking, centrifuge, or optical processing) for concentrating diseased items. The detection detector unit comprises at least one high sensitivity detector integrated onto the walls of the channels for signal detection.

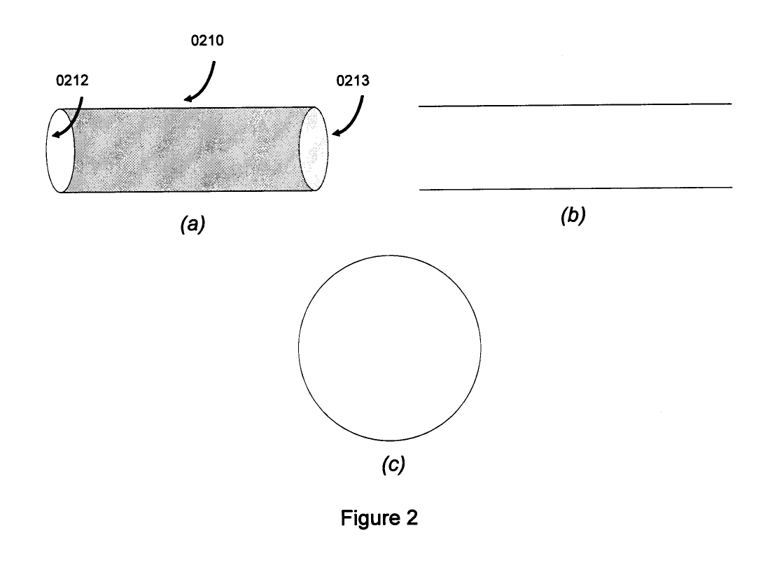

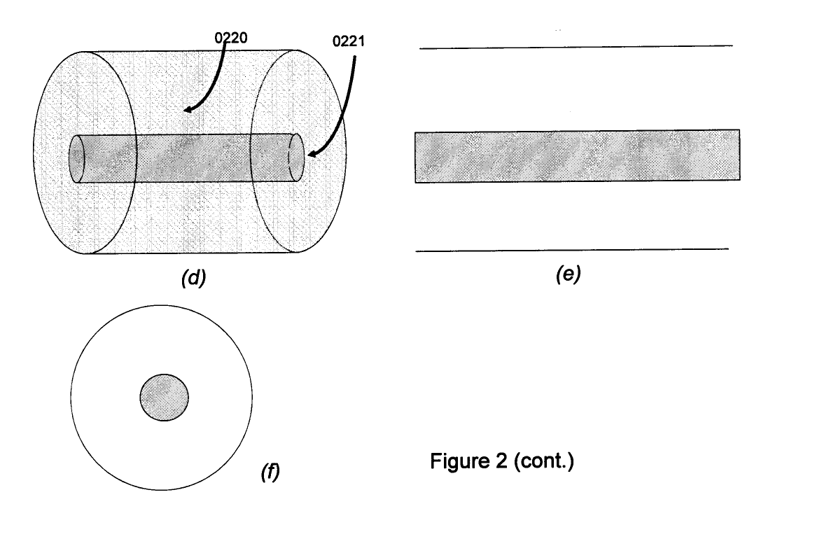

Each micro-device of this invention can further include a capillary tube having two terminal openings and a sidewall with an interior surface and an outer surface, wherein one of the two terminal openings is the inlet of the micro-device and the other terminal opening is the outlet of the micro-device. As illustrated in FIG. 2 (a), 0210 is a capillary tube with at least one inlet (0212) and at least one outlet (0213). FIG. 2 (b) is a perspective view of the capillary tube. FIG. 2 (c) is a cross-sectional view of the tube. The cross-section can be a circular, elliptical, square, rectangular, tri-angular, or polygon shape. As illustrated in FIG. 2 (d), 0220 is a capillary tube with a core 0221, and a channel is defined between the outer sidewall and the core. FIG. 2 (e) is a perspective view of the capillary tube, and FIG. 2 (f) is the vertical (cross-sectional) view.

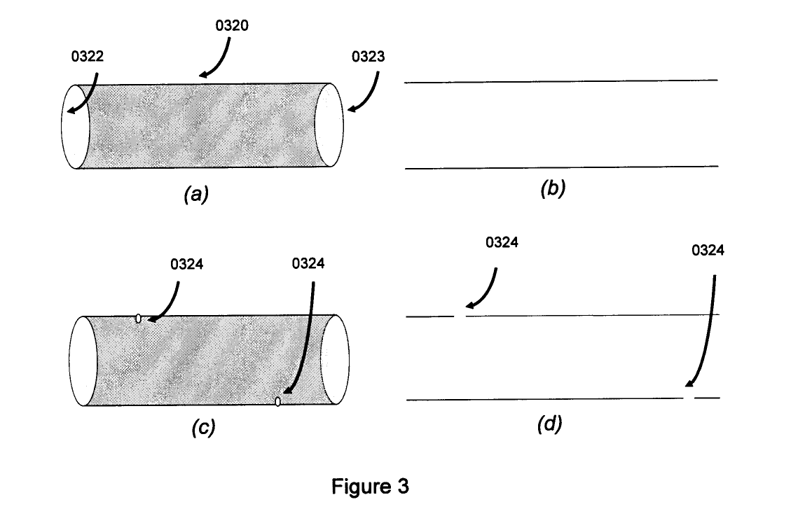

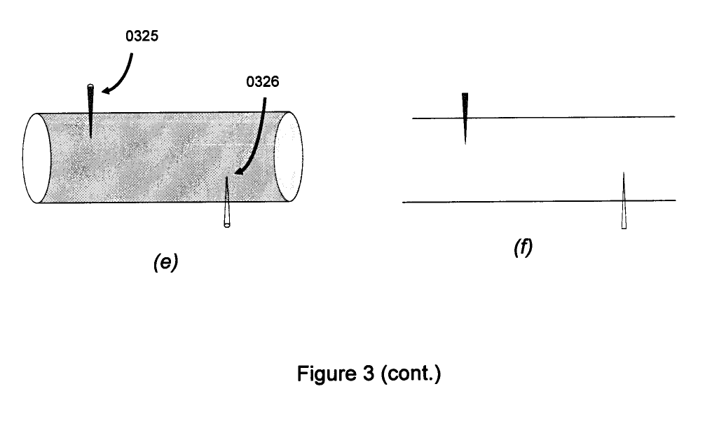

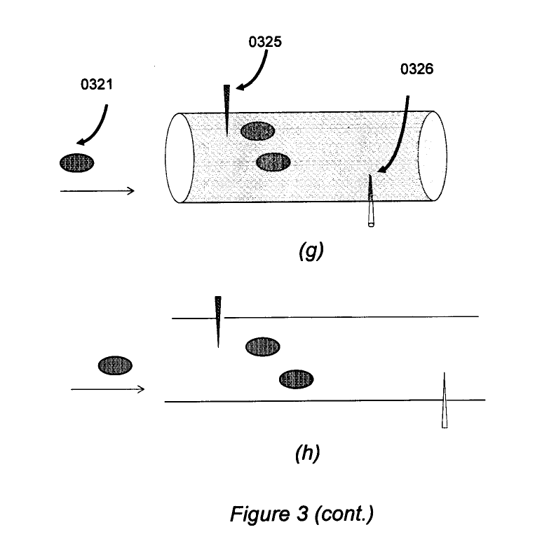

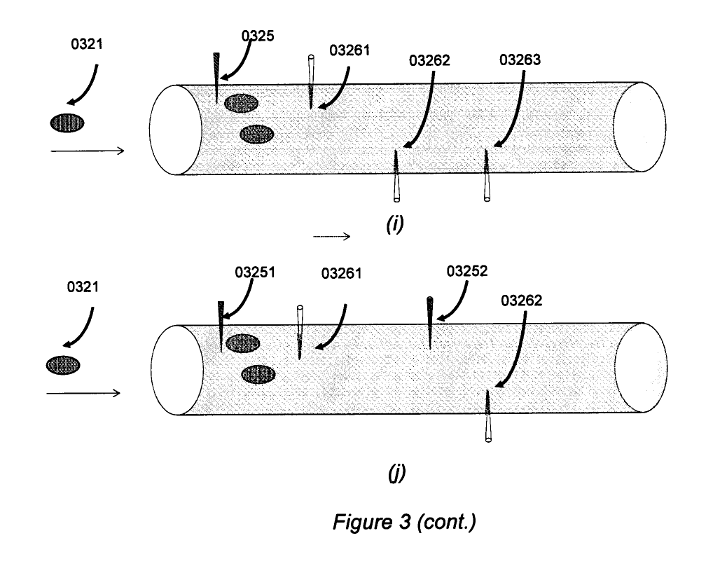

The capillary tube comprises one or more pin-holes each of which runs through the exterior and interior surfaces of the capillary tube's sidewall and houses a probing unit or a detecting unit. As illustrated in FIG. 3(a), 0320 is a capillary tube with at least one inlet (0322) and at least one outlet (0323). FIG. 3(b) is a perspective view of the capillary tube. As illustrated in FIG. 3(c), 0324 is a pin-hole which penetrates the sidewall of the capillary tube 0320. FIG. 3 (d) is a perspective view. The pin-holes can be fabricated by a mechanical, electric, magnetic, electro-magnetic, radio-active, ionic, thermal, optical, acoustical, chemical, electro-mechanical, electro-chemical, electro-chemical-mechanical method, or combination thereof.

The capillary tube can optionally be transparent. The preferred transparent materials to fabricate the capillary tube include glass, SiO₂ and organic polymeric materials. The inner diameter of the capillary tube ranges, e.g., from about 10 um to about 10 mm.

As illustrated in FIG. 3(e), a probing unit (0325) and a detecting unit (0306) are assembled penetrating the sidewall of the capillary tube. The probing unit and detecting unit are capable of sending probing signal and detecting at the microscopic level an electric, magnetic, electromagnetic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical or mechanical property of the biological subject. The probing unit is also capable of generating an electric, magnetic, electro-magnetic, radio-active, ionic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical or mechanical signal.

FIG. 3(g) illustrates an embodiment of examining tube and FIG. 3(h) is its perspective view. When the sample to be tested passes through the tube, the disturbing unit 0325 releases a pulse or disturbing signal which stimulates the sample, and then the related parameters is then probed and collected by sensor 0326. The disturbing pulse comprises an electric, magnetic, electro-magnetic, radio-active, ionic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical or mechanical signal, or a combination thereof. The probing sensor collects an electric, magnetic, electro-magnetic, radio-active, ionic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical or mechanical signal, or a combination thereof.

FIGS. 3(i) and 3(j) illustrate additional embodiments in which more than one probing unit or/and more than one detecting unit are included in the capillary tube.

Although a capillary tube is particularly exemplified herein, micro-devices containing other shapes of channels are also applicable to this invention. Such micro-devices have been previously described else by the inventors. See, e.g., WO 2012/003348 A2, WO 2012/048040, US 2010/0256518 A1, WO 2012/036697 A1, WO 2011/103041 A1, and WO 2011/005720, all of which are incorporated herein by reference in their entireties.

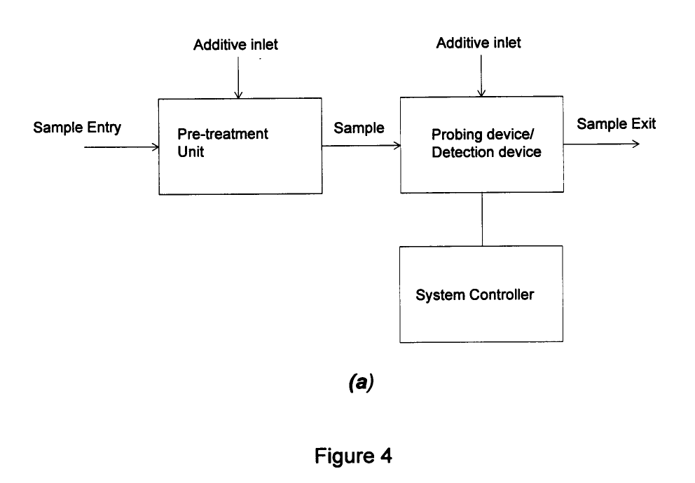

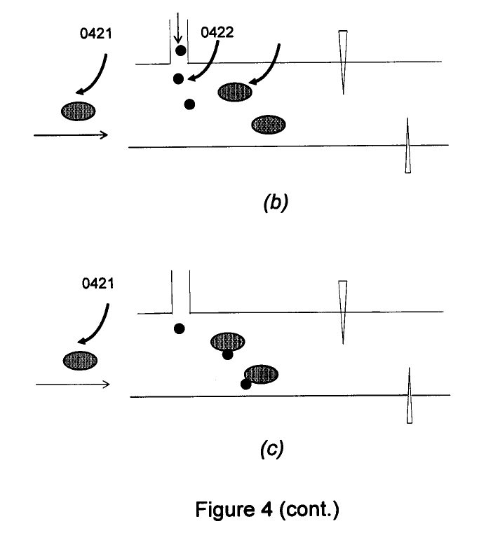

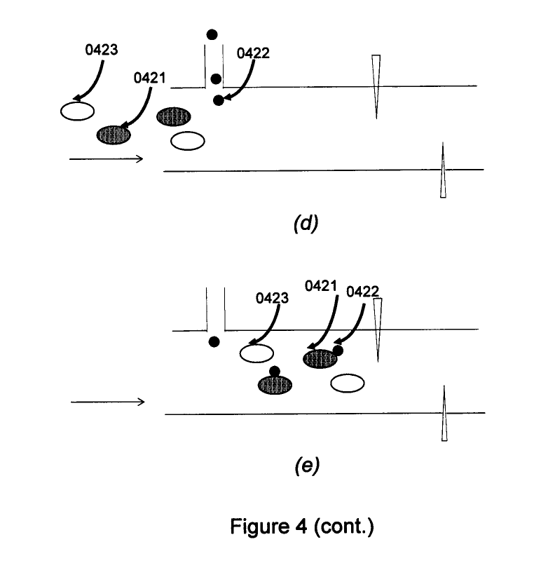

FIG. 4(a) illustrates a micro-device of this invention which includes at least a sample entry, a sample exit, an additive inlet, a pre-treat unit, a probing device, a detection device, and a system controller. In one embodiment, the additive inlet can be placed at the beginning of the process flow. For example, it can be located at the beginning portion of the pre-treatment unit. In another arrangement, multiple additive inlets can be placed at multiple locations in a machine, including at both pre-treatment unit and detection unit.

As illustrated in FIG. 4 (b), an additive 0422 can be introduced into the detection unit via additive inlet. The purpose of additive 0422 is to enhance measurement signal and therefore measurement sensitivity of biological subject 0421. In one embodiment, the additive 0422 has a higher measurement signal than that of biological subject 0421. In another embodiment, as shown in FIG. 4 (c), the additive 0422 can react with biological subject 0421 to form an aggregate, which has a higher measurement signal.

FIGS. 4(d) and 4(e) show yet another embodiment in which the additive 0422 can preferentially react with and/or absorb onto one type or types of biological subjects (biological subject 0422 in this case), thereby selectively enhancing signal from that type or types of biological subjects. For example, due to one or multiple characteristics of the said biological subject and/or additive (such as chemistry, surface properties such as chemistry and/or physical properties), the additive can react with or adsorb more strongly with one type or types of biological subjects than others. Thereby selectively enhancing measurement sensitivity of one type of types of biological subjects. One example would be a desired additive react with or adsorb more strongly with cancer cells and as a result, an enhanced or differentiated measurement signal is achieved.

The current invention is also aimed to resolve the issues encountered in the existing detection technologies and achieve the goals to carry out early stage cancer screening while still enabling identification of specific type of cancer such as bladder cancer, breast cancer, colon and rectal cancer, endometrial cancer, kidney cancer, leukemia cancer, lung cancer (including bronchus), melanoma cancer, non-Hodgkin lymphoma, pancreatic cancer, prostate cancer, thyroid cancer, with a high degree of sensitivity and specificity.

One of the keys to this innovation in meeting the above stated goals is a novel set of detection target bio-identifiers, which are novel, un-obvious, and clearly differentiated from the traditional bio-markers in terms of their specific compositions, functions, and performance. Unlike the traditional bio-markers which are typically only sensitive to one type of detection target or cancer (or even a sub-type of a cancer such as lung cancer) for each bio-marker, such detection target bio-identifiers can be used in general, early stage cancer screening with the ability to determine if there is cancer and what the specific type(s) of cancer. In terms of their compositions, the bio-identifiers used in the present invention differ from the traditional bio-marker in that they contain a set of un-obvious, novel, and more diversified groups of bio-chemical, chemical, biological, and bio-physical components. In terms of their functions, unlike traditional bio-markers which often suffer from low specificity (thus a high degree of false count) and lack of ability to probe multiple cancer types (hence not suited for general purpose cancer screening and early stage cancer screening), the bio-identifiers used in the present invention enable one to probe cancer with a high degree of both sensitivity and specificity, as well as to detect multiple cancer types with differentiated signals. Further, the micro-devices and detection methods of this invention, by utilizing the bio-identifiers, can be used in conjunction with multiple components with multiple reaction paths comprising bio-markers, samples to be detected, bio-chemicals (glucose, pyruvic acids, bromopyruvic acid, phosphoenolpyruvate (PEP), pyruvate kinase, pyruvate carboxylase, PEP carboxykinase, alanine, adenosine triphosphate, acetyl-coenzyme, oxaloacetate, lactate, ethanol, acetaldehyde, and fatty acids), chemicals (ions, catalysts, oxidizers, acidic acid, acetic acid, citric acid, tartaric acid, carcinogen, and organic components), biological components (proteins, enzymes, virus, cells, mitochondria, and power cells), and polymers. With the bio-identifiers' novel and diversified composition groups, various mechanisms and reactions including a single reaction path or multiple reaction paths can be employed to probe cancer and its type, which include but are not limited to chemical reactions (oxidation reaction, reduction reaction, exothermal reaction, and catalytic reaction), surface chemical reaction, surface bio-chemical reaction, surface physical reaction, surface physical-chemical reaction, and surface bio-physical reaction, surface adsorption and surface absorption, bio-chemical reaction, and bio-physical reaction. In terms of performance, the bio-identifiers and the methods and micro-devices of this invention are superior to those of the traditional bio-markers and have overcome most of the bio-marker's limitations, which include their inability to achieve simultaneous sensitivity and specificity, inability to detect multiple cancer types (using a given bio-marker) and hence inability to be used in general purpose cancer screening, false diagnosis (when sensitivity is high), and relatively complex process.

In contrast to traditional bio-marker of pure biological nature, the novel detection target bio-identifier disclosed in this application comprises chemical, bio-chemical, and biological Mg²⁺, H⁺, Ca²⁺, Hg²⁺, Al³⁺, NH₄⁺, H₃O⁺, Hg₂⁴⁺, Cl⁻, F⁻, Br⁻, O²⁻, CO₃²⁻, HCO₃⁻, OH⁻, NO₃⁻, PO₄³⁻, SO₄²⁻, CH₃COO⁻, HCOO⁻, C₂O₄²⁻, and CN⁻), oxidizers (e.g., O₂, O₃, H₂O₂, other inorganic oxides, F₂, Cl₂, HNO₃, nitrate compounds including ferric nitrate and silver nitrate, H₂SO₄, H₂SO₅, H₂SO₈, other persulfuric acids, chlorite, chlorate, perchlorate, other analogous halogen compounds, hypochlorite, other hypohalite compounds, NaClO, Hexavalent chromium compounds, permanganate compounds, sodium perborate, nitrous oxide, silver oxide, osmium retroxide, Tollen's reagents, 2,2′-dipyridyldisulfide (DPS), and bleach), catalysts, NaOH, KOH, CO₂, and CO. The role of the novel detection target bio-identifier is to probe biological entity to be measured to determine (a) if there is cancer in the sample and (b) what type of cancer in the sample, obtaining both measurement sensitivity for early stage cancer detection and specificity (to non-cancer components as well as specific cancers). Since the novel detection target bio-identifiers are not purely biological in nature, it avoids the major issues encountered in typical bio-marker approaches. Instead, it can both probe low level cancer signals for general purpose cancer screening, as well as diagnosing which type of cancer through sensitive differentiation between different types of cancers (comparing detected signals with stored signatures for various types of cancers).

In one embodiment, the disclosed detection target bio-identifier can enhance the measurement sensitivity of the cancer detection parameters. In other embodiment, the disclosed detection target bio-identifier can be used in conjunction of bio-marker(s). In yet another embodiment, the disclosed detection target bio-identifier can be utilized with at least one oxidizer for cancer detection. In still another embodiment, the detection target bio-identifier can be added to the sample to be measured and mixed for thorough reaction, and the sample is next centrifuged to separate out biological entities with detection target bio-identifier attached, and finally detection is carried out on separated samples. In a general application, the novel detection process disclosed in this application comprises a detection target bio-identifier, an oxidizer, a sample to be tested, a bio-marker, a chemical component, a biological component, and a bio-chemical component.

One of the roles of some of the disclosed detection target bio-identifiers is to selectively attach to cancer entities (such as a cancerous cell). Another role is to selectively attach (for other types of detection target bio-identifiers) to non-cancerous entities. Another role is to react (comprising chemically, biologically, electrically, physically, thermal, mechanically, surface chemically, surface biologically, surface physically, surface bio-chemically, bio-chemically, bio-thermally, bio-physically, bio-electrically, and electro-chemically) with the sample or certain component(s) of the sample to be tested. Yet, another important role of such detection target bio-identifier is to probe the oxygen level at a microscopic level of a biological entity to be detected through reaction with the biological entity (such as cell or protein). Such reaction can be in electric, magnetic, electromagnetic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical, or mechanical, and catalytic in nature. In addition to the above, in a general sense, the role of detection target bio-identifier is to interact and probe the biological entity being tested to extract information (such information including electric, magnetic, electromagnetic, thermal, optical, acoustical, biological, chemical, electro-mechanical, electro-chemical, electro-chemical-mechanical, bio-chemical, bio-mechanical, bio-electro-mechanical, bio-electro-chemical, bio-electro-chemical-mechanical, physical, or mechanical), and enhance measurement sensitivity as well as specificity (to differentiate between normal entity and diseased entity, as well as between different cancer types).

In one embodiment, the disclosed detection target bio-identifiers is to attach to cancer cells, while different type of cancerous cells attract different level of target bio-identifiers according to the type of the cancer, which enables the identification of the types or sub-types of the cancer, by observing and classifying target bio-identifiers' level attached to a cancer cell, and further confirm the developing stage or period of a cancer by the quantity or concentration of the cancerous cells.

In another embodiment, reaction between the detection target bio-identifier and the biological entity can go through multiple paths, including but not limited to simple chemical reaction, simple biological reaction, simple bio-chemical reaction, oxidation, reduction. It also can include catalytic reaction, complex biological reactions and bio-chemical reactions. In still another embodiment, the detection target bio-identifier can react first with another component or components (for example, a bio-marker, an ion, an oxidizer, a protein, or a catalyst) in the detection system (for example, in the detection equipment and detection chamber), and then the resultant species (from the initial reaction(s)) react or probe the biological entity being tested.

In one important embodiment, a novel detection target bio-identifier disclosed in this application (such as catalyst or ion) can react with biological entity such as cells to probe oxygen level in the biological entity to obtain macroscopic and microscopic information. The obtained oxygen level can be correlated to whether the biological entity is cancerous or not, since cancerous cell often has a lower oxygen level while normal cell has a higher oxygen level. In another embodiment, an ion and/or a catalyst can be used to interact with oxygen in the biological entity, triggering a response which can then be measured (for example, gas evolution, thermal change, and charge redistribution, etc.). With a catalytic reaction, the signal can often get enhanced even with a low level of catalyst. In still another embodiment, a desired detection target bio-identifier can react and preferentially adsorbed (or absorbed) on to a particular location in a biological entity (such as a cell), resulting in a differentiated signal (between cancerous entity and normal entity) when one or more parameters are measured on such biological entities. For example, when a Fe ion is selectively adsorbed (or absorbed) onto a biological entity (such as a cell), it will change its local electrical field and charge distribution. It may also preferentially react with certain component(s) in the biological entity and provide a differentiated signal. In other words, a detection target bio-identifier may react with and/or adsorb (or absorb) onto cancerous cell and normal cell differently, which will enhance cancer detection sensitivity and possibly specificity (target), and/or result in a differentiated signal.

As another example of cancer detection, the novel detection target bio-identifier is used to probe mitochondria respiration and oxygen level in the cell, including pyruvic acid in the biological entity since pyruvic acid is an important compound at biochemistry, and it is a key to metabolic pathways. When oxygen is insufficient, it (pyruvate) breaks down anaerobically, and energy is generated through non-oxidative breakdown of glucose, which leads to cancer. In healthy cells where oxygen level is sufficient, energy is generated from oxidative breakdown of pyruvate.

With detection target bio-identifier, one can better probe microscopic properties of the biological entity and its specific signature(s), identifying its entity (for example, which type of cell and hence which type of cancer).

In one of the embodiments, the novel detection target bio-identifier is added to the sample to be detected with some degree of selective interaction (including but not limited to attachment to certain components of the biological entity, chemical interaction, biological interaction, or bio-chemical interaction) with at least a certain component of the biological entity. Next, an alternating force or field, including but not limited to acoustic wave, optical beam, thermal wave, electrical current, electro-magnetic wave, is applied to the biological entity to be detected. Its response under this alternating force or field is then recorded. Such recorded data contains information related to the biological component (such as cancer cell) targeted by the detection target bio-identifier.

Detection of oxygen level and the detection hardware, process, and additives or bio-identifiers for cancer and other disease detection is an important innovative feature of this invention. Since it is difficult to detect low level of oxygen at a microscopic level (at DNA, RNA, protein, molecular, and cell level), novel ideas have been conceived in this patent application, in which the oxygen level is directly and indirectly measured (and calculated) using at least one detection enhancer and one micro-device. An enhancer or bio-identifier is added to the biological sample being measured and its response is then measured. The measured response can be thermal signals (for example, from exothermal reaction), physical, physical-chemical, bio-chemical (for example, bubble formation (from reaction between the added catalyst and the biological sample and its mixture), optical signal (for example, light emission, light scattering due to bubble formation, color change due to oxygen level change), chain chemical reaction due to catalytic reaction between the additive (for example, enzyme, catalyst), chemical reaction, and electrical signals (current, voltage, surface charge, permeability of ions through membrane).

In one embodiment, an oxidizer is added first to the biological sample to be tested which reacts with the sample. A second additive which could be an enzyme or a catalyst is then added next. The added enzyme or catalyst can react with the oxidized biological sample (or the sample with raised oxygen level). Various properties can next be measured using the detectors in the micro-device.

In another embodiment, a biological component such as a protein is added first, which attach preferentially to certain site(s) of one or multiple types of biological species being tested. A second additive, which is easy to be tracked and/or at least one of whose properties can be easily measured, is added to the above mentioned mixture. The solution containing the first and second additives and the biological sample to be tested is measured in the micro-device using its detection probes. Optionally, the first and the second additives are mixed first and then added to the solution containing the biological sample. Optionally, the species with the first and the second additive attached can be separated from the rest of the solution using various separation methods, and then measured.

It is another important innovative feature to use enzyme and catalyst in conjunction with micro-device (and optionally with micro-devices' geometry-dependent factors (e.g., size, shape, and material including coating material), and measure one or more properties and status of a biological sample to be tested (its optical, thermal, acoustical, chemical, physical, bio-chemical, bio-physical, mechanical, electrical, electro-magnetic properties, it size, surface area, hardness, elasticity, viscosity, and its flow speed) after the use of an enzyme or catalyst triggers a reaction or even a chain reaction (chemical, biological, and bio-chemical reactions) for enhanced response signals, which not only differentiate normal biological samples from diseased (for example, cancerous samples), but also obtain information on what type of disease (for example, what type of cancer). Sometimes it can be a one-step reaction, but it can also be a two-step or even three-step reaction.

In one embodiment, since enzymes are highly selective to substrates (for example, cell surface), the right type of enzyme selective to a given type of cancer can be used to screen that type of cancer, in conjunction of the micro-device disclosed in this application to achieve a degree of sensitivity and specificity. In another embodiment, multiple enzymes which are selective to multiple types of cancers can be used for general screening. If a patient from the above test is suspected to have a cancer, enzyme can be screened one by one to determine the type of cancer. The micro-device can be designed with multiple chambers (each chamber comprising at least one inlet for introducing at least an enzyme, a probing unit, and a detection unit) connected with one or more channels, with a biological sample being tested flowing through the chambers. As an illustration, a detection scheme using enzyme is shown below (in which the generated detection signal is detected by the micro-device, and it involves a catalytic reaction):

Enzyme+substrate(cancer surface)=>Enzyme/substrate=>Enzyme+product(can be used to detect a microscopic signal)

This present invention is particularly useful for its ability to detect disease and even differentiate different types of diseases (for example, different types of cancers). Examples of different cancer cells include carcinoma, sarcoma, leukemia, lymphoma, and glioma. Those different types of cancer cells have differences in various properties including but not limited to physical, chemical, bio-chemical, bio-physical, mechanical, thermal, optical, electrical, magnetic, and electro-magnetic properties. For example, even within the carcinoma type of cancers, squamous cells are flat on the surface, while the adenomatous type of cancers are generally bulky (thus having a lower surface area to volume ratio than the squamous type cancers). Therefore, a group of enhancers or bio-identifiers which are easily absorbs or adsorbs on the surface of cancer can be employed in conjunction with the micro-devices for achieving improved measurement specificity for squamous type of cancers, while the same group of enhancers will have lower signal strength on adenomatous type of cancers.

Another difference would be in the cell surface and cell membrane properties of different types of cancers which more specifically will have different surface properties, oxygen level, oxygen bonding within the cell and cell surface, and various permeability and transport properties. As a result, by employing enhancers which can probe oxygen level and/or bonding site, permeability properties, transport properties, and surface properties of the biological samples such as cell, protein, DNA, RNA, and tissue.

In one embodiment, an enhancer or bio-identifier containing an ion additive (e.g., Fe, Au, Ag, Cu, K, Ca, Na, and Cr) and good surface adsorption and absorption ability is utilized, which is mixed with a biological sample to be tested. The solution with the enhancer or bio-identifier and biological sample is next tested in a micro-device of this invention.

In another embodiment, an enhancer containing at least one oxidizer (such as H₂O₂) is first mixed with the biological sample to be tested. The mixed solution is then tested in the micro-device.

In yet another embodiment, an enhancer containing at least one oxidizer (such as H₂O₂) is first mixed with the biological sample to be tested. A second enhancer containing at least one catalyst is added to the above solution next. The mixed solution is then tested in the micro-device.

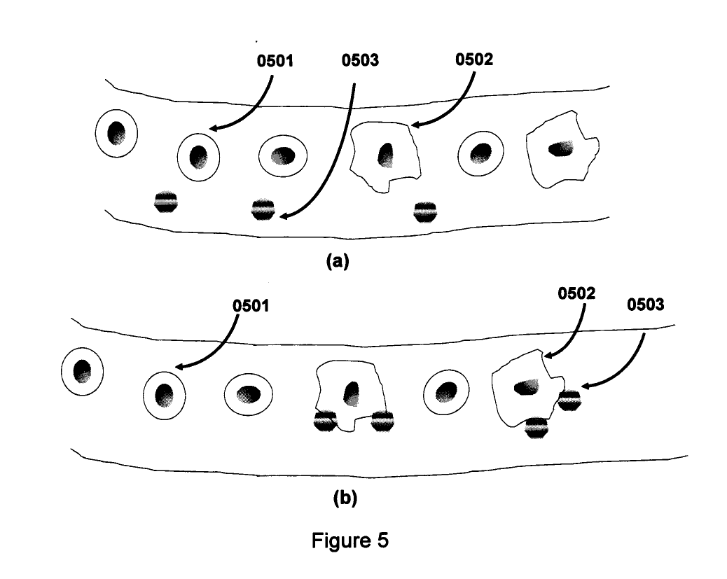

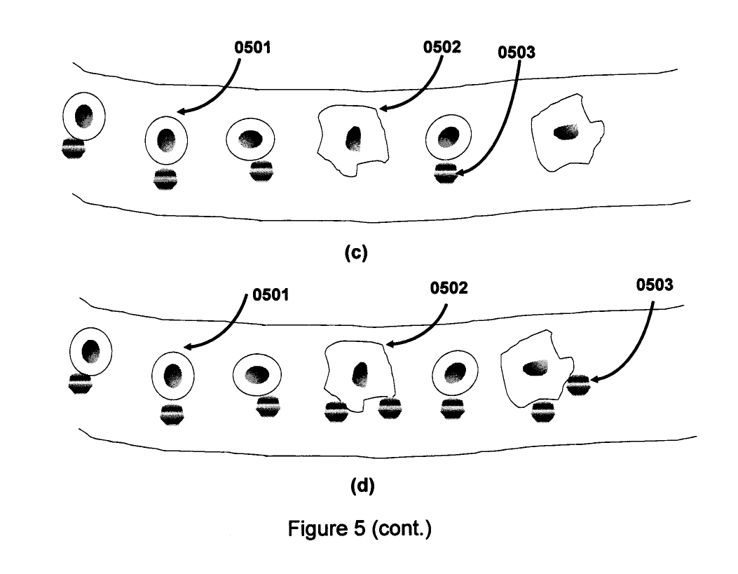

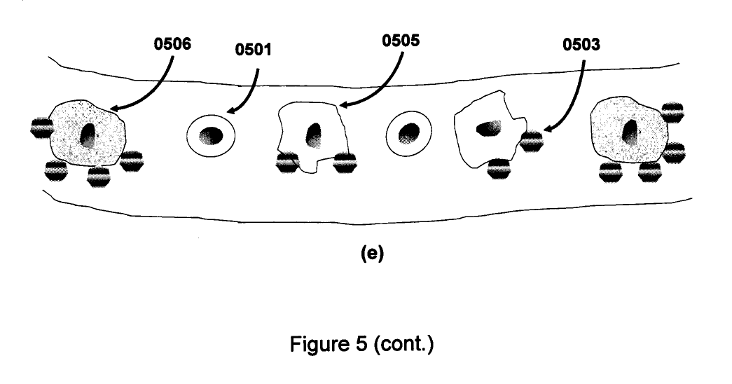

FIGS. 5-7 illustrate the principle underlying the application of the bio-identifiers used in the micro-devices and methods of this invention.

As illustrated in FIG. 5(a), 0501 is a normal cell and 0502 is a tumor cell. 0503 is a detection target bio-identifier. As illustrated in FIG. 5(b), in one embodiment, the detection target bio-identifier 0503 selectively attaches onto tumor cells 0502. With the attachment of the detection target bio-identifier onto cancerous cell saturates, the possibility of detecting and separating cancerous cells is much more enhanced.

FIG. 5(c) illustrates another embodiment in which the detection target bio-identifier selectively attaches onto normal cells, instead of attaching to cancerous cells.

FIG. 5(d) illustrates yet another embodiment in which the detection target bio-identifier attaches onto both normal cells and cancerous cells, while the attachment ratio and quantity on normal cells and cancerous cells are different, resulting in differentiated signals.

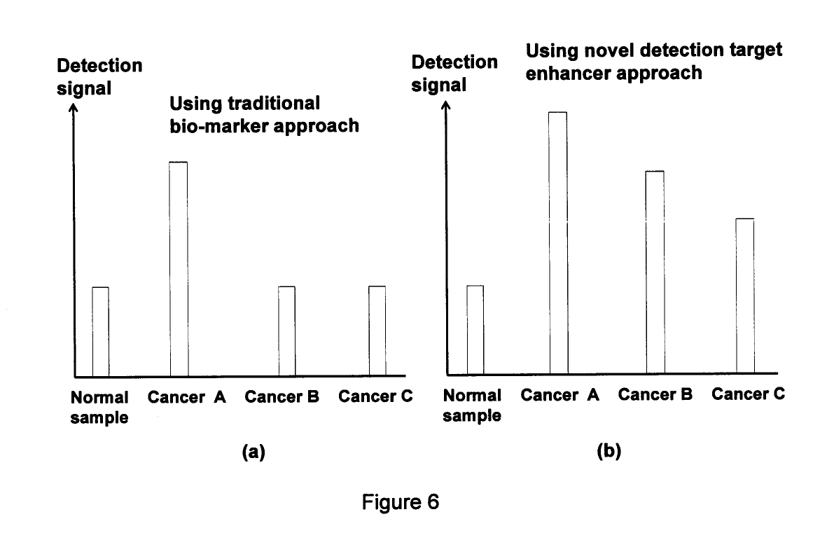

As illustrated in FIG. 5(e), 0501 is a normal cell and 0505 is tumor cell type A, and 0506 is tumor cell type B. 0503 is a detecting target bio-identifier. In one embodiment, the detection target bio-identifier selectively attaches onto cancerous cells, while attaching to different cancerous cell types at different ratio. Thus, the cancerous cell type A and cancerous cell type B can be distinguished. In addition to unobviousness and clear distinctions (relative to traditional bio-markers) of detection target bio-identifier disclosed in this application, one of the major advantages over the traditional bio-marker approach is that the innovative, new detection target bio-identifier is capable of detecting and as well as distinguishing multiple cancer types. As schematics shown in FIG. 6, while typically, traditional bio-marker can only detect one type of cancer (sometimes, even only a sub-type of one type of a cancer) (FIG. 6 (a)), the innovative detection target bio-identifier approach can not only detect multiple cancer types, it can also clearly differentiate different types of cancer, which has significant benefits in terms of applications in early and/or routine physical examinations, costs, operations, and efficiency.

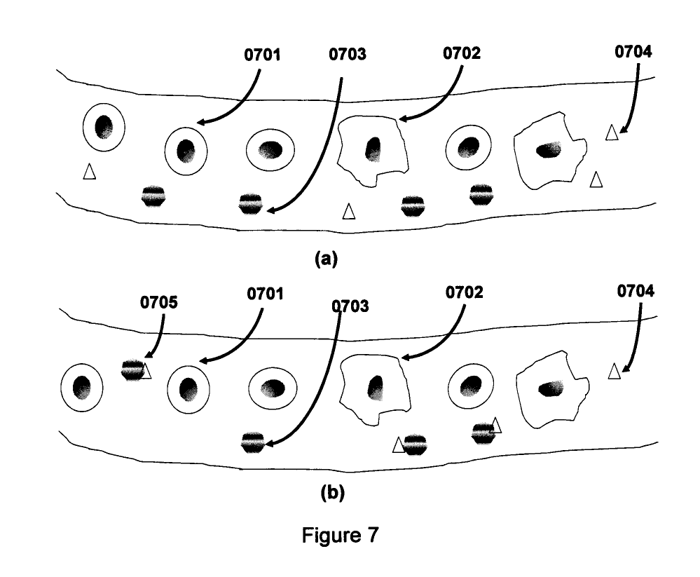

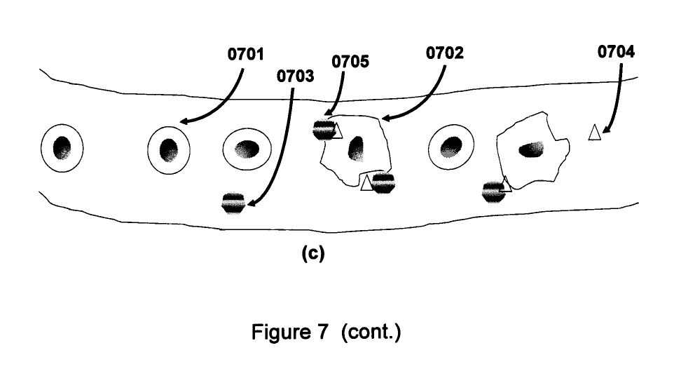

Sometimes, one or more detection target bio-identifiers (or other components such as an ion, a catalyst, an oxidizer, a protein, a chemical compound, a bio-chemical compound, or a polymer) can undergo multiple reactions, and adsorptions or absorptions before attaching or reacting to biological entities being detected. As illustrated in FIG. 7(a), 0701 is normal cell, 0702 is tumor cell, 0703 is the detection target bio-identifier C, and 0704 is an additional component D (such as a detection target bio-identifier, an ion, a catalyst, an oxidizer, a protein, an optical component like a florescence component, an radioactive component such as a positron, a chemical compound, a bio-chemical compound, or a polymer). Subsequently, the detection target bio-identifier reacts with the additional component D (0704), optionally forming a new entity 0705 as shown in FIG. 7(b). Finally, the newly formed entity 0705 selectively attach to cancer entity 0702 as shown in FIG. 7(b). Typically, the target ability and detection sensitivity of the newly formed entity 0705 is better than those of its original predecessor (0703 and 0704).

The micro-device of this invention can include multiple stages of probing or detection each provides a probing signal and detects a property at the microscopic level that can be the same as or different from the probing signal and detected property of another stage.

A multi-staged detecting device collects data in each stage and then normalizes and integrates the collected data to plot a characteristic curve to identify and diagnose the sample tested, for example, for diagnosis of the type of cancer. Specifically, among other things, at each stage, the device has a different geometry. In one embodiment, the difference in geometry of the device is the channel width and height (therefore, its cross-section). In another embodiment, the difference is in its length. In yet another embodiment, the difference is in its shape (for example, its cross-section can be circular, square, rectangular, oval and octagon). This geometrical factor, coupled with an applied probe (e.g., an optical beam, a thermal wave, a force, an acoustical wave, an electric voltage, an electronic current, or an electro-magnetic wave), and a measured response from the biological sample being measured provides information on the characteristics (a finger print) of a disease type. In one application, it provides information on the type of cancer. This geometrical factor along its properties of the biological sample being measured plays an important role in identifying the type(s) of cancer(s) in the sample. In another embodiment, the geometrical factor of the detection device, coupled with an applied probe (e.g., an optical beam, a thermal wave, a force, an acoustical wave, an electric voltage, an electronic current, or an electro-magnetic wave), at least one enhancer (including but not limited to an oxidizer, a catalyst, an enzyme, a reducing agent, an inhibitor, chemical component, a biological component, and a bio-chemical component), and a measured response from the biological sample being measured provides information on the characteristics (a finger print) of a disease type (e.g., the type of cancer). In yet another embodiment, the geometrical factor of the detection device, coupled with at least one enhancer (including but not limited to an oxidizer, a catalyst, an enzyme, a reducing agent, an inhibitor, chemical component, a biological component, and a bio-chemical component), and a measured response from the biological sample being measured provides information on the characteristics (a finger print) of a disease type (e.g., the type of cancer). In still another embodiment, the geometrical factor of the detection device, coupled with a measured response from the biological sample being measured provides information on the characteristics (a finger print) of a disease type (e.g., the type of cancer).

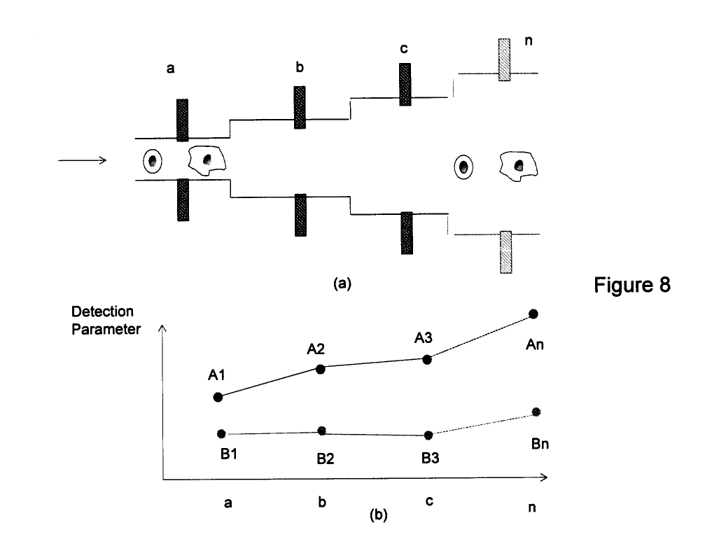

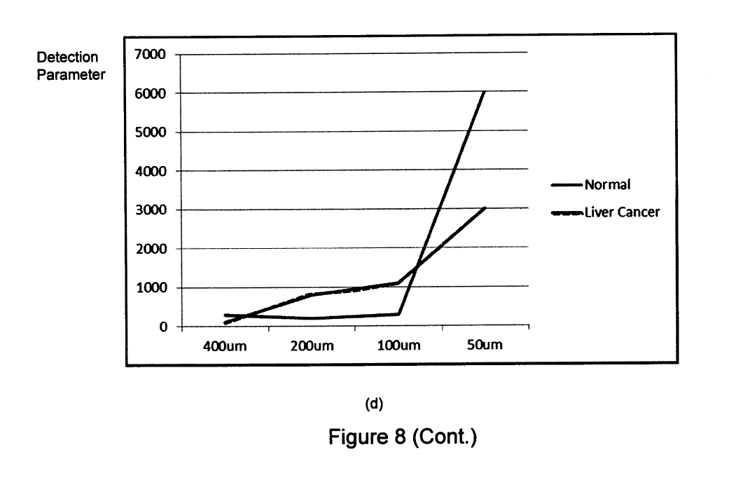

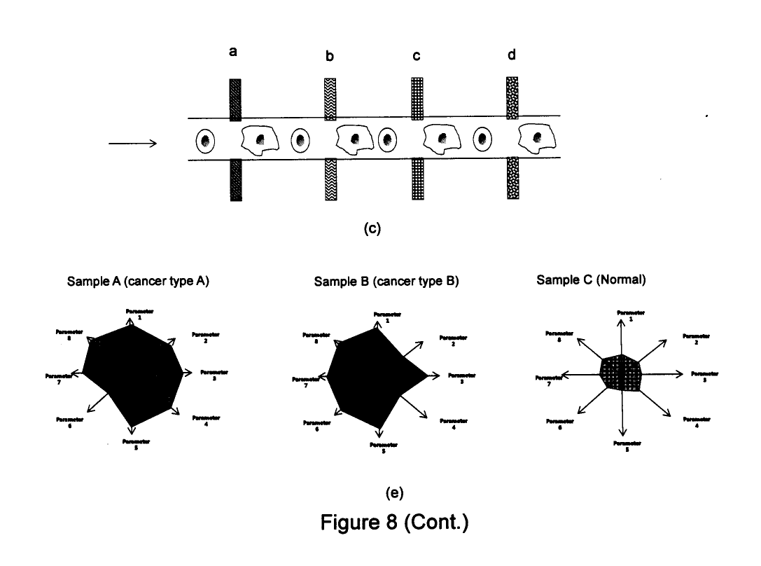

FIG. 8(a) depicts an embodiment of such devices with four different probing stages, identified as a, b, c, and n. These different stages differ from each other by the geometry of the channel—in this case the width of the channel in which the biological sample travels. In this embodiment, a biological entity is probed in all four stages and a same property is detected in each stage. The detection measurements or reading of the same property collected from the four stages can be used to plot a specific characteristic curve. See FIG. 8(b). The figure can then be used to identify the types of the tested biological sample (e.g., diseased cells) with comparison to the standing control group (e.g., non-diseased or healthy cells). In FIG. 8(b), A and B are different biological samples and result in different curves.

FIG. 8(c) illustrates another embodiment of the micro-device with multiple stages each different from the other by the probing units. The width and height of the channel in the different stages is the same. In this embodiment, two different biological samples are being probed and then detected. The measurements of the detected property can be normalized to plot a specific characteristic curve. FIG. 8(d) shows two different curves for the two different biological samples.

Multiple microscopic properties that are probed and then detected by various probing and detection units can composite a complex index, which is represented by the area inside the curves as well as the feature or shapes of the curves as shown in FIG. 8(d). This is more reliable than using a single microscopic property to determine the existence or type of disease (e.g., cancer or tumor). As shown in FIG. 8(d), cancer types A and B have different areas with regard to the standard control sample, while cancer type A and cancer type B have different features in shape as well as different contours. This provides better differentiation and identification between different types of cancers. This method allows for a multi-dimensional characterization of the tumor, and it is more sensitive and comprehensive than the traditional detection methods. Compared with traditional detection method which often only relies on one parameter or one approach for cancer detection, the method disclosed in this application which utilizes multiple parameters even including diverse properties (biological, bio-chemical, physical, bio-physical, chemical, mechanical, thermal, optical, electrical, electro-optical properties, etc.) provides much more reliable, complete, overall, accurate and sensitive information on the detection, and also improves detection specificity.

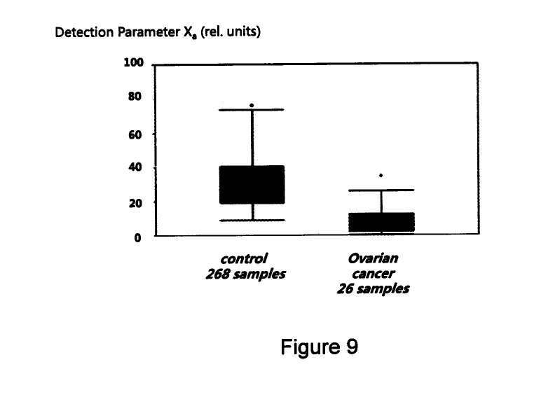

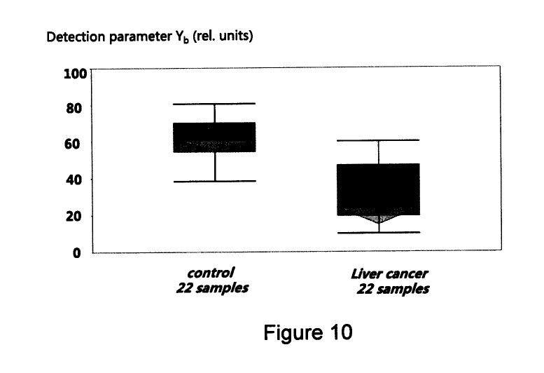

Extensive investigations were carried out to verify and confirm the utilities and applications of the invention disclosed herein. The data and results from these investigations have positively shown the effectiveness of the invention in detecting cancers and even differentiating their different types. For example, FIG. 9 and FIG. 10 show the results from using two different detection parameters disclosed in this application, with one on ovarian cancer detection utilizing detection parameter Xa and the other on liver cancer detection using detection parameter Yb. In both cases, significant differences have been observed between the control samples and cancer samples, and thus indicate and confirm the ability of the present invention to detect cancers. Sensitivity and specificity for ovarian cancer obtained from the receiver operating characteristic curve (ROC) based on the data is shown in the table below which demonstrates good sensitivity and specificity of the invention disclosed herein in detecting cancers.

| Cut-off (rel. units, for detection | | |
|---|---|---|
| parameter Xa) | Sensitivity | Specificity |
| 4 | 26.7% | 100.0% |
| 12.5 | 53.3% | 98.6% |
| 26.5 | 74.7% | 74.9% |
| 30 | 80.0% | 68.9% |
| 57.5 | 86.7% | 31.1% |
| 75 | 93.3% | 13.5% |
| 90 | 100.0% | 9.5% |

In addition to the ability to detect cancer group from the control group (normal, non-cancerous group), the investigation also showed that the invention disclosed herein could also identify specific cancer types based on their respective cut-off values and their different responses to different types of detection parameter. As another example, using detection parameter Xa, ovarian cancer showed lower cut-off value and average measured number compared with colon cancer. Therefore, it is confirmed that the invention disclosed and claimed herein can be used not only to carry out general cancer screening including early stage cancer detection to separate cancer group patients from the normal group, but also to identify and differentiate specific types of cancer.

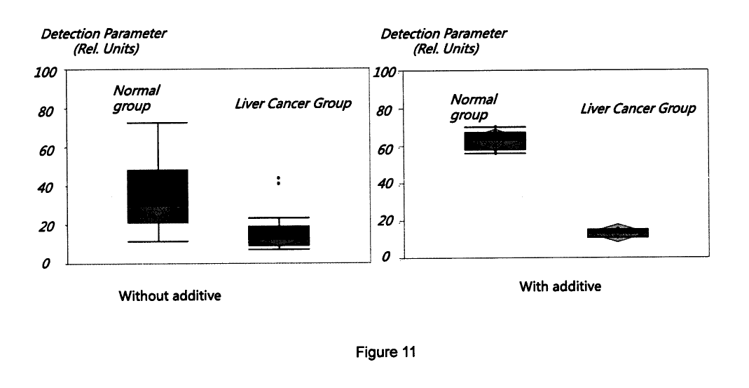

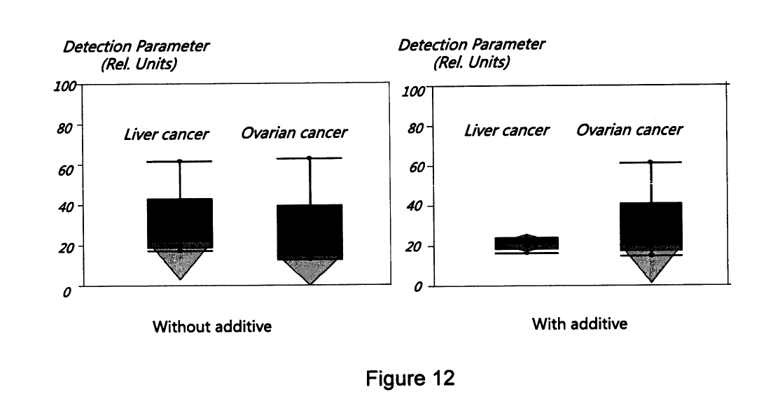

In another series of investigations, the additives disclosed herein have also shown their effectiveness in further enhancing cancer detection sensitivity and specificity Specifically, with the utilization (addition) of additives to the samples to be tested, the difference in measured signals between the normal group (non-cancerous group) and cancer group was magnified, resulting in enhanced cancer detection sensitivity and specificity. In some cases, the addition of the additives disclosed in this invention helped to identify specific cancer types, due to the observed difference in response to the additives by different cancer types. For instance, in one set of examples, liver cancer and ovarian cancer showed significantly different responses to the addition of a specific type of additive, resulting in a significant difference in signal strength for those two types of cancers. In addition to enhancing cancer detection capability (enhanced detection signal relative to normal group (healthy group)), this important feature can be effectively utilized for targeted or specific cancer screen or detection. Showing the results of two examples of these investigations are FIGS. 11 and 12. Particularly, FIG. 11 illustrates the different in detecting and identifying normal group and liver cancer group, with and without an additive, of the invention disclosed herein; while FIG. 12 shows the different detection specificity and accuracy of the invention disclosure herein for detecting and differentiating liver cancer and ovarian cancer, with and without an additive. Specifically, FIG. 11 illustrates the result of an example of the additive being used in detection. As shown in FIG. 11(a), the normal group (healthy) group and the cancer (here liver cancer) group had a gap of 16 (Rel. Units) when being tested without adding the additive X, and FIG. 11(b) shows the detection result after both the normal and liver cancer samples were mixed with a same controlled concentration of the additive X. The gap between normal group and the liver cancer group was increased by 61, about 4.7 times compared to the experiment before or without adding the additive X. The results show that the additive significantly enhanced the screening effectiveness between the normal and cancer samples. FIG. 12 shows an example of the additive being used in differentiating/discriminating and separating different types of cancer. As shown in FIG. 12(a), the liver cancer group and the ovarian cancer group had a gap of 12.5 (Rel. Units) when being tested without adding the additive Y, and FIG. 12(b) shows the detection result after both the liver cancer group and the ovarian cancer samples were mixed with a same controlled concentration of the additive Y. The gap between the liver cancer group and ovarian cancer group was increased by 84.4, or about 6.7 times, compared to the experiment before additive involved. In other words, the additive Y significantly enhanced the screening effectiveness between specified cancer.

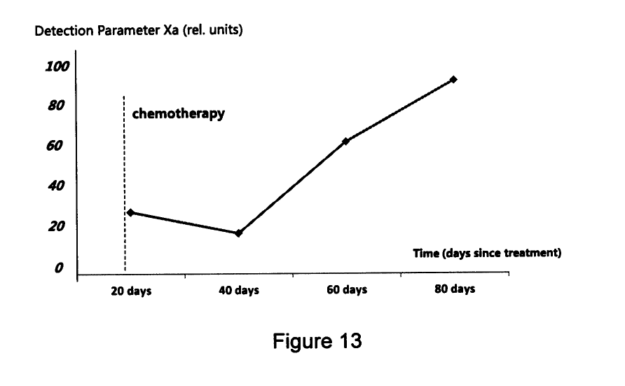

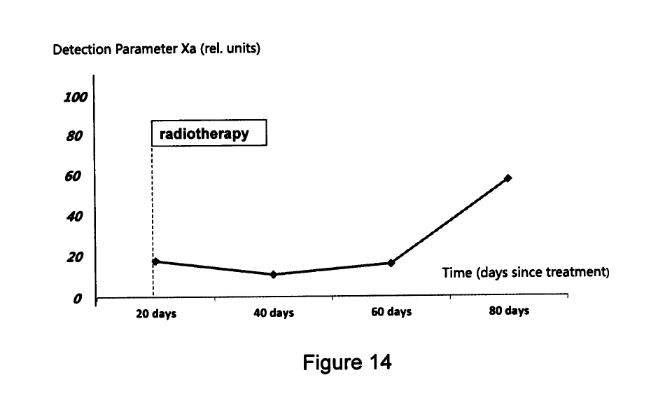

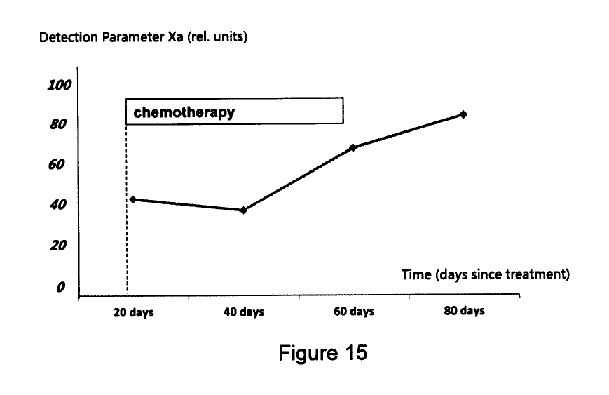

In addition, the invention disclosed herein can be used for detection of changes in the disclosed measurement properties during and post-cancer-treatment follow-up monitoring and evaluations, providing valuable assessment to the effectiveness of the treatment, patient status and guidance to follow-up treatment. FIGS. 13-15 show one of the detection parameters disclosed in this application measured in response to cancer treatment. More specifically, FIG. 13 shows the effectiveness of the invention disclosed herein in monitoring the post-chemotherapy recurrence of breast cancer; FIG. 14 shows the effectiveness of the invention disclosed herein in monitoring the post-radiotherapy recurrence of gastric cancer; and FIG. 15 shows the effectiveness of the invention disclosed herein in monitoring the post-chemotherapy recurrence of gastric cancer. In all these three figures, noticeable changes have been observed following cancer treatment, confirming the potential value of the invention disclosed herein for post-cancer-treatment monitoring.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All publications referenced herein are incorporated by reference in their entireties.

1. A micro-device for detecting at the microscopic level a property of a biological material contained in a liquid or existing in a liquid state, comprising an inlet for the biological material to enter the micro-device, an optional pre-treatment unit, a probing unit, a detection unit, a system controller, and an exit for the biological material's residue or waste to be ousted from the micro-device.

2. The micro-device of claim 1, wherein the property to be detected comprises a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanic al-optical, bio-electro-thermal-optical, bio-electro-chemical-mechanical, physical or mechanical signal, or any combination thereof.

3. The micro-device of claim 2, wherein the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility; and any of these properties can be static or dynamic and changing.

4. The micro-device of claim 1, further comprising a capillary tube having two terminal openings and a sidewall with an interior surface and an outer surface, wherein one of the two terminal openings is the inlet of the micro-device and the other terminal opening is the outlet of the micro-device, and the capillary tube houses the probing unit and optionally the detection unit.

5. The micro-device of claim 4, wherein the capillary tube comprises an interior core and an interior channel which is defined by the interior core and the interior surface of the capillary tube's sidewall, a pre-treatment unit, and a biological sample re-circulation unit.

6. The micro-device of claim 4, wherein the capillary tube comprises one or more pin-holes each of which runs through the exterior and interior surfaces of the capillary tube's sidewall and houses a probing unit or a detecting unit.

7. The micro-device of claim 4, wherein the capillary tube comprises at least two pin-holes each of which penetrates through the exterior and interior surfaces of the capillary tube's sidewall and houses a probing unit or a detecting unit.

8. The micro-device of claim 4, wherein either of the probing unit and detecting unit is capable of sending a probing signal or measuring at the microscopic level a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical signal, or a combination thereof.

9. The micro-device of claim 8, wherein the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility; and any of these properties can be static or dynamic and changing.

10. The micro-device of claim 7, wherein each pin-hole is fabricated by a method comprising mechanical, electric, magnetic, electro-magnetic, radio-active, ionic, thermal, optical, acoustical, chemical, electro-mechanical, electro-chemical, and electro-chemical-mechanical treatments.

11. The micro-device of claim 7, where each pin-hole has a diameter or width ranging from 0.01 micron to one centimeter, or from 10 microns to 2000 microns.

12. The micro-device of claim 4, wherein the capillary tube has a circular, elliptical, square, rectangular, triangular, or polygonal shape.

13. The micro-device of claim 4, wherein the capillary tube has an inner diameter or width ranging from about 0.1 um to about 10 mm, from about 20 microns to about 300 microns, from about 0.1 micro to about 100 microns, or from about 5 um to about 500 um.

14. The micro-device of claim 13, wherein the capillary tube has an a length ranging from 100 um to about 100 mm or from 100 um to about 100 mm.

15. The micro-device of claim 1, wherein the probing unit and the detection unit are embodied in one unit.

16. The micro-device of claim 1, wherein the probing unit applies a radioactive probing signal to the biological material.

17. The micro-device of claim 16, wherein the probing unit comprises a radioactive element that generates the radioactive probing signal.

18. The micro-device of claim 17, wherein the radioactive element comprises H-3, Be-7, Na-22, Ca-46, Fe-55, Fe-59, Co-56, Co-57, Co-58, Co-60, Ni-63, Zn-65, Zn-72, Kr-85, Sr-89, Sr-90, Y-90, Y-91, Zr-94, Nb-93, Nb-95, Mo-93, Ru-103, Ru-106, Ag-111, Sb-125, Te-127, Te-129, I-123, I-131, Xe-125, Xe-127, Xe-133, Cs-134, Cs-137, Ce-144, Pm-147, Eu-154, Eu-155, Ir-188, Ir-189, Ir-190, Ir-192, Ir-192, Ir-193, Ir-194, Ir-194, Pb-210, Po-210, Rn-220, Rn-222, Ra-224, Ra-225, Th-228, Th-229, Th-232, Th-234, Pa-234, Pu-238, Pu-239, Pu-240, Pu-241, Am-241, Am-242, Cm-242, Cm-243, or Cm-244.

19. The micro-device of claim 1, wherein the probing unit comprises a positron-emitting device that delivers a probing signal to the biological material, and the probing signal is a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical signal, or a combination thereof.

20. The micro-device of claim 1, wherein the detecting unit comprises a spectroscopic collector integrated to the detecting unit.

21. The micro-device of claim 1, wherein the biological material is treated or mixed with an additive and the biological material reacts or binds with the additive to form a complex, conjugate, or aggregate, thereby increasing the detection signal of the property of the biological material that is to be tested, resulting in generation and detection of new signal of at least one property of the biological material otherwise undetectable, or allowing detection of signal specific to or differentiation of cancer types.

22. The micro-device of claim 1, further comprising an additive inlet for introducing an additive to the liquid containing the biological material, wherein the addition inlet can be located before or up-stream to the inlet for the biological material to enter the micro-device, the same inlet for the biological material to enter the micro-device, located in and being part of the optional pretreatment unit, located in and being part of the probing unit, or located in or being part of the detection unit.

23. The micro-device of claim 22, wherein at least an additive inlet is connected to a pin-hole.

24. The micro-device of claim 22, wherein at least one additive inlet is located at the probing unit or the detection unit.

25. The micro-device of claim 22, wherein the additive communicates with, interacts with, or probes the biological material; or the additive triggers, participates in, or functions in a response by the biological material at the cellular level; or enhances the measurement signal of the tested property of the biological material.

26. The micro-device of claim 22, wherein the additive reacts or binds with the biological material to form a complex, conjugate, or aggregate, thereby increasing the detection signal of the property of the biological material that is to be tested, resulting in generation and detection of new signal of at least one property of the biological material otherwise undetectable, or allowing detection of signal specific to or differentiation of cancer types.

27. The micro-device of claim 22, wherein the additive selectively reacts or binds with the biological material or a component thereof, to form a complex, conjugate, or aggregate, thereby selectively increasing the strength of the property of the biological material or a component thereof that is to be tested.

28. The micro-device of claim 22, wherein the additive comprises a liquid solution, solid nanoparticles, or gas.

29. The micro-device of claim 28, wherein the liquid solution is an aqueous solution or an organic solution and comprises potassium permanganate, glucose, a glucose compound, hydrogen phosphate, pyruvate acid, sodium pyruvate, bromide pyruvate, bromopyruvic acid, acetic acid, propionaldehyde, glycerldehyde, methylglyoxal, lactate dehydrogenase, alanine, lactic acid, amino acid, a protein, calcium, potassium, sulfur, sodium, magnesium, copper, zinc, selenium, molybdenum, fluorine, chlorine, iodine, manganese, cobalt, iron, or an enzyme.

30. The micro-device of claim 29, wherein the enzyme comprises a hexokinase.

31. The micro-device of claim 28, wherein the gas comprises 0₂, 0₃, CO, CO₂, calcium, sodium, potassium, sulfur, sodium, magnesium, copper, zinc, selenium, molybdenum, fluorine, chlorine, iodine, manganese, cobalt, iron, or carbon based organics.

32. The micro-device of claim 22, wherein the additive comprises an ion, an oxidant, a reductant, an inhibitor, a catalysts, an enzymes, a bio-marker, a chemical-marker, a bio-chemical marker, a bio-active compound, a chemical component, a bio-chemical component, a biological component, an organic component, a metal-organic component, a bio-chemical component, an optical components, a florescence component, a protein, a virus, a coloring agent, an antibody, or a combination thereof.

33. The micro-device of claim 32, wherein the ion comprises Fe³⁺, Fe²⁺, Ag⁺, Cu²⁺, Cr³⁺, Na⁺, K⁺, Pt²⁺, Mg²⁺, H⁺, Ca²⁺, Hg²⁺, Al³⁺, NH₄⁺, H₃O⁺, Hg₂⁴⁺, Cl⁻, F⁻, Br⁻, O²⁻, CO₃²⁻, HCO₃⁻, OH⁻, NO₃⁻, PO₄³⁻, SO₄²⁻, CH₃COO⁻, HCOO⁻, C₂O₄²⁻, or CN⁻; the oxidant comprises oxygen, ozone, hydrogen peroxide, an inorganic peroxide, nitric acid, a nitrate compound, a chromium compound, a permanganate compound, sulfuric acid, persulfuric acid, fluorine, chlorine, bromine, iodine, chlorite, chlorate, perchlorate, a halogen compound, hyperchlorite, a hypohalite compound, sodium perborate, nitrous oxide, sliver oxide, osmium tetroxide, Tollens' reagent, 2,2′-dipyridyldisulfide, urea, or a combination thereof; the reductant comprises nascent hydrogen, a compound containing Fe²⁺ ion, sodium amalgam, sodium borohydride, a sulfite compound, hydrazine, a compound containing the Sn²⁺ ion, zinc-mercury amalgam, lithium aluminum hydride, Lindlar catalyst, formic acid, oxalic acid, ascorbic acid, phosphites, hypophosphites, or phosphorous acid; or the bio-active compound comprises glucose, fructose, pyruvate, galactose, amino acid, acetic acid, glyoxylic acid, oxalic acid, propionic acid, acetic acid, or an enzyme.

34. The micro-device of claim 1, wherein the detection unit detects the property at the microscopic level and generates a machine-readable tested signal.

35. The micro-device of claim 34, wherein the system controller processes the machine-readable signal to generate data that can be displayed or readable by human eyes.

36. The micro-device of claim 35, wherein the system controller comprises a first A/D converter, a computer, and a data display device.

37. The micro-device of claim 36, wherein the system controller further comprises an amplifier which amplifies the machine-readable tested signal before it reaches the A/D converter and then the computer.

38. The micro-device of claim 36, wherein the computer comprise a CPU, a RAM, and a ROM.

39. The micro-device of claim 36, wherein the system controller further comprises a manipulator which initiates or generates a disturbing signal and then sends it to the computer for processing before the disturbing signal is applied to the biological material by the disturbing unit.

40. The micro-device of claim 36, wherein the disturbing signal is a thermal, optical, acoustical, biological, chemical, radioactive, electrical, magnetic, electro-mechanical, electro-chemical, electro-optical, electro-thermal, electro-magnetic, electro-chemical-mechanical, bio-chemical, bio-mechanical, bio-optical, bio-thermal, bio-physical, bio-electro-mechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical or mechanical signal, or a combination thereof.

41. The micro-device of claim 40, wherein the electrical property is surface charge, surface potential, resting potential, electrical current, electrical field distribution, electrical dipole, electrical quadruple, three-dimensional electrical or charge cloud distribution, electrical properties at telomere of DNA and chromosome, capacitance, or impedance; the thermal property is temperature or vibrational frequency; the optical property is optical absorption, optical transmission, optical reflection, optical-electrical property, brightness, or fluorescent emission; the chemical property is pH value, chemical reaction, bio-chemical reaction, bio-electro-chemical reaction, reaction speed, reaction energy, speed of reaction, oxygen concentration, oxygen consumption rate, oxygen bonding site, oxygen bonding strength, local charge density due to oxygen atom and/or molecule properties and locations, local ionic density due to oxygen atom and/or molecule properties and locations, local electric field density due to oxygen atom and/or molecule properties and locations, ionic strength, catalytic behavior, chemical additives to trigger enhanced signal response, bio-chemical additives to trigger enhanced signal response, biological additives to trigger enhanced signal response, chemicals to enhance detection sensitivity, bio-chemicals to enhance detection sensitivity, biological additives to enhance detection sensitivity, or bonding strength; the physical property is density, shape, volume, or surface area; the biological property is surface shape, surface area, surface charge, surface biological property, surface chemical property, pH, electrolyte, ionic strength, resistivity, cell concentration, property relating to a bio-marker, or biological, electrical, physical or chemical property of solution; the acoustic property is frequency, speed of acoustic waves, acoustic frequency and intensity spectrum distribution, acoustic intensity, acoustical absorption, or acoustical resonance; the mechanical property is internal pressure, hardness, flow rate, viscosity, shear strength, elongation strength, fracture stress, adhesion, mechanical resonance frequency, elasticity, plasticity, or compressibility; and
any of these properties can be static or dynamic and changing.

42. The micro-device of claim 36, wherein the system controller further comprises a second A/D converter and a signal generator which process the disturbing signal after the computer but before the disturbing signal is applied to the biological material by the disturbing unit.

43. The micro-device of claim 1, wherein the pre-treatment unit separates the biological material into different components by the difference in a common property of the biological material, treats the surface of biological subject to be tested, or mixes the biological subject to be tested with at least one additive.

44. The micro-device of claim 1, further comprising a second optional pre-treatment unit, a second probing unit, and a second detection unit, which form a second stage of detection within the micro-device, wherein the second stage of detection detects the same or different property at the microscopic level as the previous stage.

45. The micro-device of claim 44, wherein the second stage has a different geometry than the first stage.

46. The micro-device of claim 45, wherein the geometry is the width, height, length, or shape of the channel, or a combination thereof.

47. The micro-device of claim 1, wherein the geometry information, probing signals applied by the probing units, the signals detected and measured by the detection units, or/and the optionally use of one or more enhancers, results in enhanced specificity and sensitivity of the detection and differentiation of different types of disease.

48. The micro-device of claim 1, wherein the different types of disease are different types of cancer.

49. The micro-device of claim 1, wherein the different types of cancer include bladder cancer, breast cancer, colon cancer, rectal cancer, ovarian cancer, endometrial cancer, kidney cancer, liver cancer, gastric cancer, leukemia cancer, lung cancer, melanoma cancer, non-Hodgkin lymphoma, pancreatic cancer, prostate cancer, or thyroid cancer.
